(19) United States
(12) Patent Application Publication
(10) Pub. No.: US 2015/0174157 A1
(43) Pub. Date: Jun. 25, 2015

(54) INHALED NO DONOR PIPERAZINYL DERIVATIVE PREVENTING ALLERGIC PULMONARY VASCULAR AND BRONCHIAL INFLAMMATION BY REDUCING VEGF AND RESTORING eNOS IN HYPOXIC PULMONARY ARTERY

(71) Applicant: KAOHSIUNG MEDICAL UNIVERSITY, KAOHSIUNG (TW)

(72) Inventor: Ing-Jun CHEN, KAOHSIUNG (TW)

(73) Assignee: KAOHSIUNG MEDICAL UNIVERSITY, KAOHSIUNG (TW)

(21) Appl. No.: 14/634,440

(22) Filed: Feb. 27, 2015

Related U.S. Application Data
(63) Continuation-in-part of application No. 13/437,550, filed on Apr. 2, 2012.
Application No. 13/437,550 is a continuation-in-part of application No. 13/095,393, filed on Apr. 27, 2011, now abandoned.
Application No. 13/095,393 is a continuation-in-part of application No. 12/572,519, filed on Oct. 2, 2009, now abandoned.
Application No. 12/572,519 is a continuation-in-part of application No. 11/857,483, filed on Sep. 19, 2007, now abandoned.

(30) Foreign Application Priority Data
Jun. 15, 2007 (TW) 96121950

(51) Int. Cl. A61K 31/785; A61K 31/519; A61K 31/455; A61K 31/717; A61K 31/78; A61K 31/375; A61K 31/201; A61K 31/522; A61K 31/351

(52) U.S. Cl. CPC A61K 31/785; A61K 31/522; A61K 31/519; A61K 31/455; A61K 31/351; A61K 31/78; A61K 31/375; A61K 31/201; A61K 31/717

(57) ABSTRACT

A method for treating a disease is disclosed. The method includes steps of providing a subject in need thereof; and administering one selected from a group consisting of Piperazinyl Analogs and Piperazinyl Complex Analogs compound represented by formula II or formula III, a pharmaceutically acceptable salts thereof; and a pharmaceutical composition thereof to the subject in a dosage between 1 and 5.0 milligrams per kilogram of body weight, in a liquid mist, dry powder or aerosolized formulation.

This application is a continuation-in-part of application Ser. No. 13/437,550 filed on Apr. 2, 2012, which is a continuation-in-part of application Ser. No. 13/095,393 filed on Apr. 27, 2011, which is a continuation-in-part of application Ser. No. 12/572,519 filed on Oct. 2, 2009, which is a continuation of application Ser. No. 11/857,483 filed on Sep. 19, 2007, for which priority is claimed under 35 U.S.C. sctn. 120; and this application claims priority of application No. 96121950 filed in Taiwan on Jun. 15, 2007 under 35 U.S.C. sctn. 119; the entire contents of all are hereby incorporated by reference.

### FIELD OF THE INVENTION

The present invention relates to a combination therapy method of a pharmaceutical composition of a Piperazinyl derivative and additional active agents capable of preventing allergic pulmonary vascular inflammation and remodeling, and is useful for the treatment of asthma and respiratory obstruction disease.

### BACKGROUND OF THE INVENTION

Inflammatory processes are prominent in chronic obstructive pulmonary disease (COPD) and hypoxic pulmonary artery hypertension and are increasingly recognized as major pathologic components of pulmonary vascular remodeling. Despite the clinical and functional consequences of peri-bronchial micro-vascular remodeling in asthmatic lungs, up to now, there is little data on therapeutic approaches to this phenomenon.

Anti-inflammatory therapies in the airway are rather ineffective at improving chronic symptoms and reducing inflammation, or reversing the lung function decline and airway remodeling that accompanies reduced functioning of the pulmonary vascular system. Specific drugs directed against vascular remodeling and chronic inflammations are needed to prevent lung tissue damage and a progressive lung function decline.

Previously, xanthine-based KMUP-1 has been proved to relax vascular and airway smooth muscle contractions by enhancing endothelial nitric oxide synthase/guanosine 3′,5′-cyclic monophosphate (eNOS/cGMP)-pathway, including increasing soluble guanylyl cyclase al (sGCα1) and protein kinase G (PKG) expression (Lin R J, et al., 2006, Wu B N, et al., 2006; Wu B N, et al., 2011; Liu C P, et al., 2012). Potentially releasing NO gas from epithelium or endothelium to smooth muscles of airway and pulmonary artery.

Sildenafil has shown anti-inflammatory action against COPD and hypoxic pulmonary artery hypertension, including reducing airway hyper-reactivity, leucocyte influx and elevated NO; its effectiveness was confirmed by measuring elevated S-notroso-N-acetylpenicillamine-induced cGMP generation (Toward T J, et al., 2004, Alp S, et al., 2006). The benefits of sildenafil encouraged us to search for even more effective eNOS/cGMP-enhancers, such as KMUPs derivative and KMUPs-RX complex compound, to inhibit COPD and hypoxic pulmonary artery hypertension.

Endothelial and eNOS/cGMP-signaling is involved in the control of lung function and cell growth. Rapid release of nitric oxide (NO) from the endothelium and epithelium may influence adjacent smooth muscle cell contractility and growth. Endogenous NO produced by activation of eNOS can act as a signaling molecule in several processes, including regulation of pulmonary vascular and airway smooth muscle tone via NO releasing.

Overexpression of eNOS suppresses the features of allergic asthma, originally characterized by expression of inflammatory inducible nitric oxide synthase (iNOS) and exhalation of a moment of NO from the airway. In addition, bronchial constriction can cause a pulmonary hypoxic state, reducing vascular endothelial eNOS and decreasing vascular density.

### SUMMARY OF THE INVENTION

In accordance with an aspect of the present invention, a method for inhibiting a pulmonary inflammation is disclosed. The method includes steps of providing a subject suffering from the pulmonary inflammation; and administering a pharmaceutical composition of a KMUPs complex compound selected from a group consisting of KMUPs-RX complex compound, KMUPs-RX-RX complex compound to the subject in a dosage from 1 to 5.0 milligram per kilogram of body weight for animals.

In accordance with another aspect of the present invention, a method for treating a disease is disclosed. The method includes steps of providing a subject in need thereof; and administering one selected from a group consisting of Sildenafil Analogs derivative compound and Sildenafil Analogs-RX complex compound, a pharmaceutically acceptable salts thereof; and a pharmaceutical composition thereof to the subject in an oral dosage form of 1 to 5.0 milligram per kilogram of body weight for animals.

In accordance with another aspect of the present invention, a method for treating a disease is disclosed. The method includes a step of administering a therapeutically effective amount of a compound being one selected from a group consisting of Piperazinyl Analogs and Piperazinyl Complex Analogs to a mammal having the disease including one selected from a group consisting of an obstructive pulmonary disease, a pulmonary artery hypertension, pulmonary artery proliferation, an allergic pulmonary vascular inflammation, a vascular remodeling inhibiting disease and a combination thereof.

In accordance with a further aspect of the present invention, a method for treating a disease is disclosed. The method includes a step of administering a therapeutically effective amount of pharmaceutical composition, in which the active agent is one selected from a group consisting of a Sildenafil Analogs derivative compound and a Sildenafil complex compound.

A further aspect for the combination is to administer an effective amount of a KMUPs derivative compound and other additional active agents to a mammal in need thereof.

A further aspect for the combination is to administer a therapeutically effective amount of a pharmaceutical composition, in which the active agent is a Sildenafil Analogs compound and additional active agents to a mammal in need thereof.

The pulmonary inflammatory disease-inhibiting pharmaceutical composition includes:
an effective amount of a KMUPs complex compound represented by formula II or formula III, wherein
R₂ and R₄ are each selected independently from the group consisting of a C1˜C5 alkoxy group, a hydrogen, a nitro group, and a halogen atom;
RX includes a carboxylic group which donated from one of a Statin analogues, a Co-polymer, a poly-γ-polyglutamic acid derivative, an D-ascorbic acid, L-ascorbic acid, DL-ascorbic acid, an oleic acid, a phosphoric acid, a citric acid, a nicotinic acid and a sodium CMC; and
⁻RX is an anion of a carboxylic group donated from one of a statins analogues, a Co-polymer, a poly-γ-polyglutamic acid derivative, an D-ascorbic acid, L-ascorbic acid, DL-ascorbic acid, an oleic acid, a phosphoric acid, a citric acid, a nicotinic acid and a sodium CMC; and
a pharmaceutically acceptable carrier.

KMUP-1 (1.0˜2.5 mM, 30 mins) dose-dependently increases eNOS and decreases MMP-9 expression in mice lung tissues; KMUP-1 shows marked reductions of inflammatory cells, increasing iNOS and MMP-9 and decreases sGCα1 and PKG expressions; KMUP-1 nebulization significantly reduces the increase in total cells, lymphocytes and eosinophils elicited in the airway lumen 24 hrs after the last ovalbumin challenge; inhaled NO donors KMUPs derivatives prevent allergic pulmonary vascular inflammation; a combination treatment or prevention of Allergic Pulmonary Vascular Inflammation is taken as an example.

The above objects and advantages of the present invention will become more readily apparent to those ordinarily skilled in the art after reviewing the following detailed descriptions and accompanying drawings, in which:

### BRIEF DESCRIPTION OF THE DRAWINGS

The patent or application file contains at least one drawing executed in color. Copies of this patent or patent application publication with color drawings will be disclosed by the Office upon request and payment of the necessary fee.

FIG. 1 shows the animal chamber of chronic hypoxia.

FIGS. 2A-2D show the recording of pulmonary artery blood pressure (PABP).

FIG. 2A shows the pulmonary artery blood pressure of normoxia (20% O₂) treated with a vehicle for 21 days.

FIG. 2B shows the pulmonary artery blood pressure of hypoxia (10% O₂) treated with a vehicle for 21 days.

FIG. 2C shows the normoxia treated with KMUP-1 and hypoxia with KMUP-1-ascorbate complex.

FIG. 2D shows the normoxia treated with Sildenafil and hypoxia with Sildenafil-ascorbate complex.

FIG. 3A-3D show the morphologic demonstration of a cross-section of pulmonary artery caused by long-term hypoxia and treatments.

FIG. 3A shows the normoxia (20% O₂) treated with a vehicle for 21 days.

FIG. 3B shows the hypoxia (10% O₂) treated with vehicle for 21 days.

FIG. 3C shows the effects of the KMUP-1[Hypoxia with KMUP-1-ascorbate complex (5 mg/kg/day)] for 21 days.

FIG. 3D shows the effects of the Sildenafil [Hypoxia with Sildenafil-ascorbate complex (5 mg/kg/day)] for 21 days.

FIG. 4A-4D show the morphologic demonstration of a cross-section of heart caused by long-term hypoxia and treatments.

FIG. 4A shows the normoxia (20% O₂) treated with a vehicle for 21 days.

FIG. 4B shows the effects of the KMUP-1[Hypoxia with KMUP-1-ascorbate complex (5 mg/kg/day)] for 21 days.

FIG. 4C shows the hypoxia (10% O₂) treated with a vehicle for 21 days.

FIG. 4D shows the effects of the Sildenafil [Hypoxia with Sildenafil-ascorbate complex (5 mg/kg/day)] for 21 days.

FIGS. 5A-5B show the effects of relative wall thickness of pulmonary artery and weight ratio of heart hypertrophy.

FIG. 5A shows the effect of relative wall thickness of pulmonary artery:

1, Normoxia;

2, Hypoxia;

3, Hypoxia+KMUP-1-ascorbate complex; and

4, Hypoxia+Sildenafil-ascorbate complex.

FIG. 5B shows the effect of weight ratio of RV/LV+S:

1, Normoxia;

2, Hypoxia;

3, Hypoxia+KMUP-1-ascorbate complex; and

4, Hypoxia+Sildenafil-ascorbate complex.

FIGS. 6A-6D show the effects of pulmonary brown immunohistochemistry of hypoxia endothelium, wherein (+) indicates thick immunostaining and (−) indicates abated immunostaining reactivity 20×.

FIG. 6A shows the effect on rats which were exposed to normoxia (20% O₂).

FIG. 6B shows the effect on rats which were exposed to hypoxia (10% O₂).

FIG. 6C shows the effects of the KMUP-1 [Hypoxia with KMUP-1-ascorbate complex (5 mg/kg/day)] for 21 days.

FIG. 6D shows the effects of the Sildenafil [Hypoxia with Sildenafil-ascorbate complex (5 mg/kg/day)] for 21 days.

FIGS. 7A-7D show the effects of pulmonary brown immunohistochemistry of hypoxia endothelium and smooth muscle, wherein (+) indicates thick immunostaining and (−) indicates abated immunostaining reactivity 20×.

FIG. 7A shows the effect on rats which were exposed to normoxia (20% O₂).

FIG. 7B shows the effect on rats which were exposed to hypoxia (10% O₂).

FIG. 7C shows the effects of the KMUP-1 [Hypoxia with KMUP-1-ascorbate complex (5 mg/kg/day)] for 21 days.

FIG. 7D shows the effects of the Sildenafil [Hypoxia with Sildenafil-ascorbate complex (5 mg/kg/day)] for 21 days.

FIGS. 8A-8D show the effects of pulmonary brown immunohistochemistry of hypoxia endothelium and treatment, wherein (+) indicates thick brown immunostaining and (−) indicates abated immunostaining reactivity 20×.

FIG. 8A shows the effect on rats which were exposed to normoxia (20% O₂).

FIG. 8B shows the effect on rats which were exposed to hypoxia (10% O₂).

FIG. 8C shows the effects of the KMUP-1 [Hypoxia with KMUP-1-ascorbate complex (5 mg/kg/day)] for 21 days.

FIG. 8D shows the effects of the Sildenafil [Hypoxia with Sildenafil-ascorbate complex (5 mg/kg/day)] for 21 days.

FIGS. 9A-9D show the effects of brown immunohistochemistry of hypoxia endothelium, smooth muscle and treatment, wherein (+) indicates thick brown immunostaining and (−) indicates abated immunostaining reactivity 20×.

FIG. 9A shows the effect on rats which were exposed to normoxia (20% O₂).

FIG. 9B shows the effect on rats which were exposed to hypoxia (10% O₂).

FIG. 9C shows the effects of the KMUP-1 [Hypoxia with KMUP-1-ascorbate complex (5 mg/kg/day)] for 21 days.

FIG. 9D shows the effects of the Sildenafil [Hypoxia with Sildenafil-ascorbate complex (5 mg/kg/day)] for 21 days.

FIGS. 10A-10D show the effects of long-term hypoxia-induced eNOS, sGCα1, PKG and PDE-5A expression of lung tissue:

1, Normoxia;

2, Hypoxia;

3, Hypoxia+KMUP-1-ascorbate complex; and

4, Hypoxia+Sildenafil-ascorbate complex.

FIG. 10A shows the effects of long-term hypoxia-induced eNOS expression.

FIG. 10B shows the effects of long-term hypoxia-induced sGCα1 expression.

FIG. 10C shows the effects of long-term hypoxia-induced PKG expression.

FIG. 10D shows the effects of long-term hypoxia-induced PDE-5A expression.

FIGS. 11A-11B show the effects of long-term hypoxia-induced ROCKII and VEGF expression of lung tissue:

1, Normoxia;

2, Hypoxia;

3, Hypoxia+KMUP-1-ascorbate complex; and

4, Hypoxia+Sildenafil-ascorbate complex.

FIG. 11A shows the effects of long-term hypoxia-induced ROCKII expression.

FIG. 11B shows the effects of long-term hypoxia-induced VEGF expression.

FIGS. 12A-12D show the effects of pulmonary artery expression of ROCKII, sGCα and VEGF after short-term hypoxia.

FIG. 12A shows the effects on rats which were exposed to normoxia (20% O₂).

FIG. 12B shows the effects of ROCKII expression.

FIG. 12C shows the effects of sGCα expression.

FIG. 12D shows the effects of VEGF expression.

FIG. 13 shows the effect of pulmonary NOx production:

1, Normoxia;

2, Hypoxia;

3, Hypoxia+KMUP-1-ascorbate complex; and

4, Hypoxia+Sildenafil-ascorbate complex.

FIG. 14 shows the effect of fluorescence of ROS production:

1, Normoxia;

2, Hypoxia;

3, Hypoxia+KMUP-1-ascorbate complex; and

4, Hypoxia+Sildenafil-ascorbate complex.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; they is not intended to be exhaustive or to be limited to the precise form disclosed.

In accordance with an aspect of the present invention, Piperazinyl Analogs and Piperazinyl Complex Analogs are classified into two categories, consisting of KMUPs Complex and Sildenafil Analogs Complex. KMUPs complex compounds are one of the active agent, consisting of KMUPs derivative and an RX group that can be designated by the general formula KMUPs-RX. Another active agent of the Sildenafil Analogs complex is known as the general formula Sildenafil Analogs-RX.

In an embodiment of the present invention, a combination therapy is disclosed for treating and/or preventing pulmonary disease. The compositions are formulated and administered in the manner as detailed herein. The KMUPs derivative compounds represented by the structural formula I may be used effectively alone or in combination with one or more active agents depending on the desired target therapy. Combination therapy includes administration of a single pharmaceutical dosage composition which contains a compound of structural formula I and one or more additional active agents, as well as other administration types of additional active agents, and each active agent has its own separate pharmaceutical dosage formulation.

When used in combination, in some embodiments, the compound of structural formula I may be administered as a single pharmaceutical dosage composition that contains additional active agents. In other embodiments, separate dosage compositions are administered; the formula I compound and the other additional pharmaceutical agents may be administered at essentially the same time, for example, concurrently, or at separately staggered times, for example, sequentially. In certain examples, the individual components of the combination may be administered separately, at different times during the course of therapy, or concurrently, in divided or single combination forms. Also disclosed is, for example, simultaneous, staggered and alternating treatment.

For example, a compound of structural formula I can be administered to the patient together in a single oral dosage composition such as a tablet or capsule, or each agent administered in separate oral dosage formulations. Where separate dosage formulations of additional active agents are used, can be administered other administration type at essentially the same time. An example of combination treatment may be by any suitable administration route including oral (including buccal and sublingual), rectal, nasal, airway inhalation (e.g., liquid mist, dry powder or aerosolized formulation), vaginal, and parenteral (including subcutaneous, intramuscular, intravenous and intradermal), topical administration include, but are not limited to, sprays, mist, aerosols, solutions, lotions, gels, creams, ointments, pastes, unguents, emulsion and suspensions, with oral or parenteral being preferred. The preferred route may vary with the condition and age of the recipient.

While it is possible for the administered ingredients to be administered alone, it is preferable to present them as part of a pharmaceutical formulation. The formulations of the present invention comprise the administered ingredients, as defined above, together with one or more acceptable carriers and optionally other additional therapeutic ingredients. The carrier(s) must be “acceptable” in the sense of being compatible with the other ingredients of the formulation and not deleterious to the recipient.

According to the above-mentioned aspect of the present invention, additional active agents may include additional compounds according to the invention, or one or more other pharmaceutically active agents. In preferable embodiments, the inventive compositions will contain the active agents, including the inventive combination of therapeutic agents, in an amount effective to treat an indication of interest.

Preferably, in one embodiment, additional active agents consist of Statin analogues, a Co-polymer, a poly-γ-polyglutamic acid derivative, D-ascorbic acid, L-ascorbic acid, DL-ascorbic acid, oleic acid, phosphoric acid, a citric acid, a nicotinic acid and a sodium carboxyl methylcellulose (sodium CMC).

The substance being distributed is suitably administered in any way in which at least some (preferably about 1 wt. % to about 100 wt. %) of the substance reaches the bloodstream of the organism. Thus, the substance can be administered enterally (via the alimentary canal) or parenterally (via any route other than the alimentary canal, such as, e.g., through intravenous injection, subcutaneous injection, intramuscular injection, inhalation percutaneous application, etc.).

According to a further feature of this aspect of the invention there is disclosed a method for producing a treating effect of pulmonary disease in a warm-blooded animal, such as man, in need of such treatment which comprises administering to said animal an effective amount of Piperazinyl Analogs and Piperazinyl Complex Analogs, or a pharmaceutical acceptable salt, solvate, solvate of such a salt or a prodrug thereof.

A pharmaceutical composition is disclosed in the present application, in which the active agent is a theophylline-based moiety compound for treating a pulmonary disease, and a KMUPs derivative compound has the activity of released NO, including cGMP-dependent and cGMP-independent activities.

A theophylline-based moiety compound, i.e. KMUPs derivative, which is obtained by reacting a theophylline compound with a piperazine compound and then recrystallizing the intermediate therefrom, is disclosed in the present invention. The KMUPs derivative compound has effects for treating a pulmonary disease and the benefits of good solubility, low toxicity and safety.

Preferably, The pharmaceutical composition includes one of a KMUPs derivative compound having formula I or its salts,

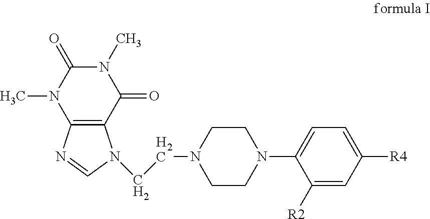

wherein R₂ and R₄ are each selected independently from the group consisting of a C1˜C5 alkoxy group, a hydrogen, a nitro group, and a halogen atom. The above-mentioned halogen refers to fluorine, chlorine, bromine and iodine.

Preferably, in one embodiment, the compound of formula I is KMUP-1, wherein R₂ is chlorine atom and R₄ is hydrogen, which has the general chemical name 7-[2-[4-(2-chlorophenyl)piperazinyl]ethyl]-1,3-dimethyl xanthine. The compound of formula I is KMUP-2, wherein R₂ is a methoxy group and R₄ is hydrogen, which has the chemical name 7-[2-[4-(2-methoxybenzene)piperazinyl]ethyl]-1,3-dimethylxanthine. In another embodiment, the compound of formula I is also KMUP-3, wherein R₂ is hydrogen and R₄ is a nitro group, which has the chemical name 7-[2-[4-(4-nitrobenzene)piperazinyl]ethyl]-1,3-dimethylxanthine. In another embodiment, the compound of formula I is also KMUP-4, wherein R₂ is a nitro group and R₄ is hydrogen, which has the chemical name 7-[2-[4-(2-nitrobenzene)piperazinyl]ethyl]-1,3-di methylxanthine.

The present invention discloses a KMUPs complex compound having a formula II of KMUPs-RX or formula III of KMUPs-RX-RX,

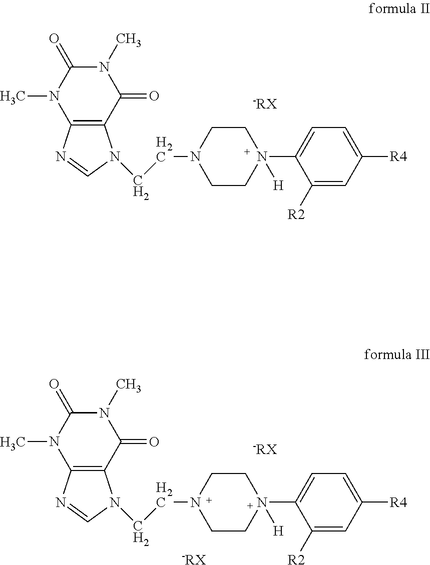

wherein R₂ and R₄ are each selected independently from the group consisting of a C1˜C5 alkoxy group, a hydrogen, a nitro group, and a halogen atom;
RX includes a carboxylic group which donated from one of a Statin analogues, a Co-polymer, a poly-γ-polyglutamic acid derivative, an D-ascorbic acid, L-ascorbic acid, DL-ascorbic acid, an oleic acid, a phosphoric acid, a citric acid, a nicotinic acid and a sodium CMC; and
⁻RX is an anion of a carboxylic group donated from one of a statins analogues, a Co-polymer, a poly-γ-polyglutamic acid derivative, an D-ascorbic acid, L-ascorbic acid, DL-ascorbic acid, an oleic acid, a phosphoric acid, a citric acid, a nicotinic acid and a sodium CMC.

Preferably, the above statins analogues are commercially available statin derivative drugs, including Atorvastatin, Cerivastatin, Fluvastatin, Lovastatin, Mevastatin, Pravastatin, Rosuvastatin, Simvastatin and Pramastatinic acid. A Co-polymer is one selected from a group consisting of a hyaluronic acid, a polyacrylic acid, a polymethacrylates (PMMA), an Eudragit, a dextran sulfate, a heparan sulfate, a polylactic acid or polylactide (PLA), a polylactic acid sodium (PLA sodium) and a polyglycolic acid sodium (PGA sodium). A Poly-γ-polyglutamic acid (γ-PGA) derivative is one selected from a group consisting of an alginate sodium, a poly-γ-polyglutamic acid sodium (γ-PGA sodium), a poly-γ-polyglutamic acid (γ-PGA) and an alginate-poly-lysine-alginate (APA). Hyaluronic Acid is a polymer, which is composed of alternating units of N-acetyl glucosamine (NAG) and D-glucuronic acid. Eudragit is a trade name for a series of copolymers derived from the esters of acrylic and methacrylate acid.

The term “KMUPs derivative compound” as used herein refers to one selected from a group consisting of KMUP-1, KMUP-2, KMUP-3, KMUP-4 and its pharmaceutical acceptable salts. Preferably, the pharmaceutical composition further includes at least one of a pharmaceutically acceptable carrier and an excipient.

To achieve the above purpose, formula I salts, formula II and formula III can be synthetically produced from the 2-Chloroethyltheophylline compound and piperazine substituted compound.

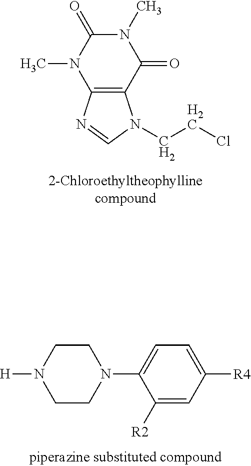

The compounds of formula I salts and KMUPs complex compound set forth in the examples below were prepared using the following general procedures as indicated.

The general procedure 1 includes steps of dissolving 2-Chloroethyl theophylline and piperazine substituted compound in a hydrous ethanol solution, and the amount of reagent should be conjugated depending on the molecular weight percentage. After adding a strong base e.g. sodium hydroxide (NaOH) or sodium hydrogen carbonate (NaHCO₃) to make the solution more alkaline or more basic, a heating procedure is performed under reflux for three hours. Allowed to stand overnight, the cold supernatant was decanted solvents were efficiently removal by vacuum concentration, and then the residue was dissolved with a one-fold volume of ethanol and a three-fold volume of 2N hydrochloric acid (HCl), kept at 50° C. to 60° C. to make a saturated solution (pH 1.2). The saturated solution was sequentially treated, decolorized with activated charcoal, filtered, deposited overnight and filtered to obtain KMUP-1 HCl with a white crystal.

The general procedure 2 includes steps of dissolving 2-Chloroethyl theophylline and piperazine substituted compound in a hydrous ethanol solution, and the amount of reagent should be conjugated depending on the molecular weight percentage. Then, a heating procedure was performed under reflux for three hours. Allowed to stand overnight, the cold supernatant was decanted solvents were efficiently removal by vacuum concentration, and then the residue was dissolved with a one-fold volume of ethanol and a three-fold volume of 2N hydrochloric acid (HCl), kept at 50° C. to 60° C. to make a saturated solution (pH 1.2). The saturated solution was sequentially treated, decolorized with activated charcoal, filtered, deposited overnight and filtered to obtain KMUP-1 HCl with a white crystal.

According to the general procedure 1 or 2, KMUPs derivatives of formula I can be synthetically produced directly, from the 2-Chloroethyl-theophylline compound and piperazine substituted compound. Preferably, a theophylline-based moiety compound derivative, i.e. KMUPs derivative, which is obtained by reacting a theophylline compound with a piperazine compound and then recrystallizing the intermediate therefrom, is disclosed in the present invention.

Preferably, the pharmaceutical acceptable salts of KMUP-1, KMUP-2, KMUP-3 and KMUP-4 are citric acid, nicotinic acid and hydrochloride.

Thereby, a KMUPs compound represents a KMUPs complex compound. Preferably, in one embodiment, KMUP-1 is dissolved in a mixture of ethanol and γ-Polyglutamic acid. The solution is reacted at warmer temperature, the methanol is added thereto under room temperature, and the solution is incubated overnight for crystallization and filtrated to obtain KMUP-1-γ-Polyglutamic complex. According to the above-mentioned general procedure 1 or 2, a sufficient amount of KMUPs derivative reacts with a carboxyl group of “RX” to form a formula II of the KMUPs complex compound. Further, the term “RX” group can refer to the Statin analogues, Co-polymer, poly-γ-polyglutamic acid derivative, D-ascorbic acid, L-ascorbic acid, DL-ascorbic acid, oleic acid, phosphoric acid, citric acid, nicotinic acid or sodium CMC, which contains a sufficient amount of the carboxyl group and can react with the piperazine group of KMUPs derivative to prepare the above formula III of the KMUPs complex according to the method in the present invention. The synthesized KMUPs complex compound shows a pro-drug and multiple therapeutic functions in the body via a chemical or an enzymatic hydrolysis. In formula II, the KMUPs complex compound is also known as a KMUP-RX complex compound and formula III is also represented as a KMUP-RX-RX complex compound.

Specifically speaking, the KMUPs-RX complex compounds, in one embodiment, as various KMUPs derivatives: include a KMUP-1-Atorvastatin complex compound, KMUP-2-Atorvastatin complex compound, KMUP-3-Atorvastatin complex compound, KMUP-4-Atorvastatin complex compound, KMUP-1-Cerivastatin complex compound, KMUP-2-Cerivastatin complex compound, KMUP-3-Cerivastatin complex compound, KMUP-4-Cerivastatin complex; KMUP-1-Fluvastatin complex compound, KMUP-2-Fluvastatin complex compound, KMUP-3-Fluvastatin complex compound, KMUP-4-Fluvastatin complex compound; KMUP-1-Lovastatin complex compound, KMUP-2-Lovastatin complex compound, KMUP-3-Lovastatin complex compound, KMUP-4-Lovastatin complex; KMUP-1-Mevastatin complex compound, KMUP-2-Mevastatin complex compound, KMUP-3-Mevastatin complex compound, KMUP-4-Mevastatin complex compound, KMUP-1-Pravastatin complex compound, KMUP-2-Pravastatin complex compound, KMUP-3-Pravastatin complex compound, KMUP-4-Pravastatin complex compound, KMUP-1-Rosuvastatin complex compound, KMUP-2-Rosuvastatin complex compound, KMUP-3-Rosuvastatin complex compound, KMUP-4-Rosuvastatin complex compound, KMUP-1-Simvastatin complex compound, KMUP-2-Simvastatin complex compound, KMUP-3-Simvastatin complex compound, KMUP-4-Simvastatin complex; KMUP-1-ascorbic acid complex compound, KMUP-2-ascorbic acid complex compound, KMUP-3-ascorbic acid complex compound, KMUP-4-ascorbic acid complex; KMUP-1-phosphoric acid complex compound, KMUP-2-phosphoric acid complex compound, KMUP-3-phosphoric acid complex compound, KMUP-4-phosphoric acid complex; KMUP-1-CMC complex compound, KMUP-2-CMC complex compound, KMUP-3-CMC complex compound, KMUP-4-CMC complex compound, KMUP-1-hyaluronic complex compound, KMUP-2-hyaluronic complex compound, KMUP-3-hyaluronic complex compound, KMUP-4-hyaluronic complex compound, KMUP-1-polyacrylic complex compound, KMUP-2-polyacrylic complex compound, KMUP-3-polyacrylic complex compound, KMUP-4-polyacrylic complex compound, KMUP-1-Eudragit complex compound, KMUP-2-Eudragit complex compound, KMUP-3-Eudragit complex compound, KMUP-4-Eudragit complex; KMUP-1-polylactide complex compound, KMUP-2-polylactide complex compound, KMUP-3-polylactide complex compound, KMUP-4-polylactide complex; KMUP-1-polyglycolic complex compound, KMUP-2-polyglycolic complex compound, KMUP-3-polyglycolic complex compound, KMUP-4-polyglycolic complex compound, KMUP-1-dextran sulfate complex compound, KMUP-2-dextran sulfate complex compound, KMUP-3-dextran sulfate complex compound, KMUP-4-dextran sulfate complex compound, KMUP-1-heparan sulfate complex compound, KMUP-2-heparan sulfate complex compound, KMUP-3-heparan sulfate complex compound, KMUP-4-heparan sulfate complex compound, KMUP-1-alginate complex compound, KMUP-2-alginate complex compound, KMUP-3-alginate complex compound, KMUP-4-alginate complex compound, KMUP-1-γ-PGA complex compound, KMUP-2-γ-PGA complex compound, KMUP-3-γ-PGA complex compound, KMUP-4-γ-PGA complex compound, KMUP-1-APA complex compound, KMUP-2-APA complex compound, KMUP-3-APA complex compound and KMUP-4-APA complex compound etc.

In accordance with a further aspect of the present invention, depending on the desired clinical use and the effect, the adaptable administration method for the KMUPs derivative and KMUPs complex pharmaceutical composition includes one selected from a group consisting of an oral administration, an intravenous injection, a subcutaneous injection, an intraperitoneal injection, an intramuscular injection and a sublingual administration.

Preferably, in another embodiment, the term “Sildenafil Analogs compound” as used herein refers to one selected from a group consisting of, Sildenafil, Hydroxyhomosildenafil, Desmethylsildenafil, Acetidenafil, Udenafil, Vardenafil and Homosildenafil. The compound Sildenafil has the chemical name 5-[2-etthoxy-5-(4-methylpiperazin-1-yl-sulphonyl)phenyl]-1-methyl-3-n-propyl-1,6-dihydro-7H-pyrazolo[4,3-d]pyrimidin-7-one, or 1-[[3-(4,7-dihydro-1-methyl-7-oxo-3-propyl-1H-pyrazolo[4,3-d]pyrimidin-5-yl)-4-ethoxyphenyl]sulfonyl]-4-methylpiperazine. The compound HydroxyhomoSildenafil has the chemical name 1-[[3-(6,7-dihydro-1-methyl-7-oxo-3-propyl-1H-pyrazolo[4,3-d]pyrimidin-5-yl)-4-ethoxy-phenyl]sulfonyl]-4-hydroxyethyl-piperazine. The compound Desmethylsildenafil has the chemical name 5-[2-ethoxy-5-(1-piperazinylsulfonyl)-phenyl]-1,6-dihydro-1-methyl-3-propyl-7H-pyrazolo[4,3-d]pyrimidin-7-one. The compound Acetidenafil has the chemical name 5-{2-ethoxy-5-[2-(4-ethylpiperazine-1-yl)-acetyl]pheny 1}-1-methyl-3-n-propyl-1,6-dihydro-7H-pyrazolo[4,3-d]pyrimidin-7-one. The compound Udenafil has the chemical name 5-[2-propyloxy-5-(1-methyl-2-pyrollidinylethylamido-sulfonyl)phenyl]-1-methyl-3-propyl-1,6-dihydro-7H-pyrazolo[4,3-d]pyrimidine-7-one. The compound Vardenafil has the chemical name 2-[2-ethoxy-5-(4-ethyl-piperazin-1-yl-sul fonyl)phenyl]-5-methyl-7-propyl-1H-imidazo[5,1-f][1,2,4]triazin-4(3H)-one. The compound HomoSildenafil has the chemical name 5-[2-ethoxy-5-[(4-ethyl-1-piperazinyl)sulfonylphenyl]-1,6-dihydro-1-methyl-3-propyl-7H-pyrazolo[4,3-d]pyrimidin-7-one.

Sildenafil Analogs derivative compounds compound

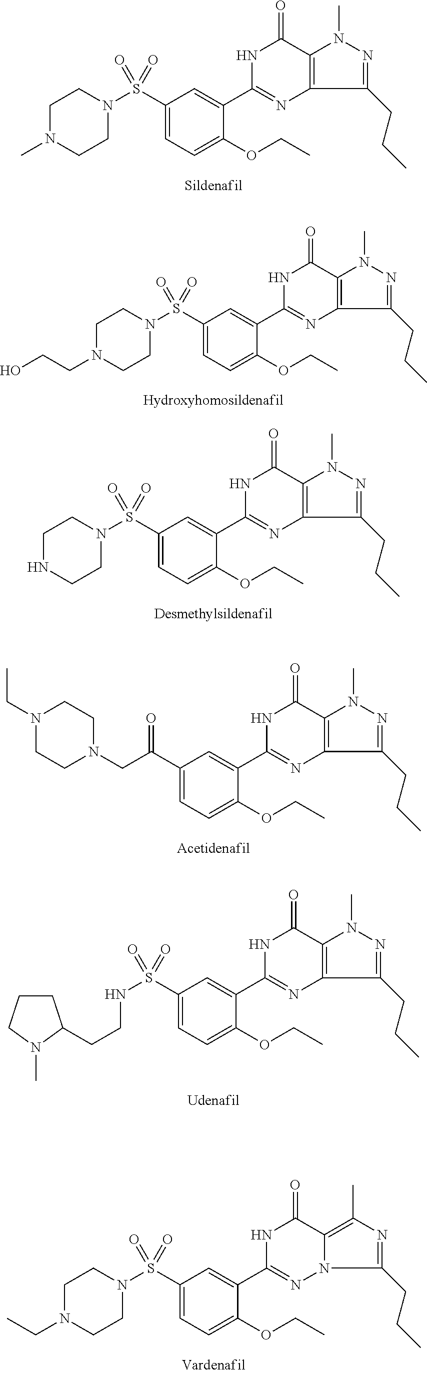

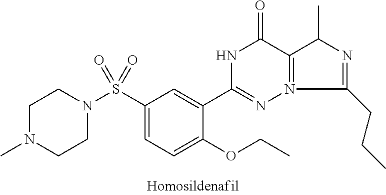

Preparation of Sildenafil analogs complex compounds from Sildenafil citrate and Sildenafil

Sildenafil analogs complex compound also designated by the general formula Sildenafil Analogs-RX complex compound, were prepared according to the abovementioned general procedure. Preferably, in an embodiment, crude Sildenafil citrate is blended and suspended in a sodium hydroxide solution to dissolve the component. After filtration of the sodium citrate, the precipitated Sildenafil base and added to an equal molecular-weight of ascorbic acid which was dissolved in methanol to react at 50° C. After overnight cooling in a glass flask, a white precipitate is obtained by filtration and then re-crystallized as Sildenafil ascorbate from ethanol.

In another embodiment, Sildenafil citrate is dissolved in diluted water, adjusted with HCl solution to pH 7.0 and separated into an ethyl acetate fraction to remove the citric acid, a hydrochloride and a sodium chloride into water fraction. The obtained Sildenafil base in ethyl acetate is dried under a de-pressurized condition. Then one carboxylic group of the RX and Sildenafil base is dissolved in methanol to react at 50° C. After sitting overnight, a white precipitate is obtained by filtration and then recrystallized as Sildenafil complex compounds from ethanol.

In another embodiment, Sildenafil HCl and sodium carboxylate of the RX are dissolved in methanol at an equal molecular weight to react at 50° C. After overnight cooling, a white precipitate is obtained by filtration and then recrystallized as Sildenafil complex compounds from ethanol. However, based on the abovementioned procedure, Sildenafil analogs derivatives complex compounds can be prepared selectively with one of the hydrochloride salts of Sildenafil analogs derivatives and one of the RX group.

Preferably, in one embodiment, Piperazinyl Analogs and Piperazinyl Complex Analogs compound aerosol formulations also use for administration. The use of aerosols to administer medicaments has been known for several decades. Such aerosols generally comprise the medicament, one or more propellants and either a surfactant or a solvent, such as ethanol. The majority of available pressurized metered-dose inhaler (pMDI) devices contain chlorofluorocarbon (CFC) propellants, which have well-documented adverse effects on the atmospheric ozone layer. Over the past few years, research has led to the development and approval of hydrofluoroalkane (HFA)-based aerosols, which do not have ozone-depleting properties, as alternatives to CFC propellants. In another embodiment, liquid mist may be delivered in a gaseous mixture containing oxygen, for example, 20% oxygen or more, as in inspired air. Alternatively, the mist may be delivered in a gaseous mixture containing increased fractions of oxygen, for example, more than 20% oxygen or more. The remaining inspired gas can include one or more gaseous fluorinated compound (any of those described herein, such as light fluorocarbons, hydrofluorocarbons or hydrochlorofluorocarbons) rather than nitrogen to increase the cooling capacity of the gaseous mixture, thus further reducing the amount of liquid fluorocarbon required. Other possible components of the gaseous mixture include, but are not limited to, nitrogen, CO₂, as present in carbogen, helium, etc. The fluorinated gas might also be SF₆, a substance approved for many other indications in humans.

Accordingly, the present invention relates to suspensions of crystalline tiotropium bromide monohydrate in the propellant gases HFA 227 (1,1,1,2,3,3,3-heptafluoropropane) and/or HFA 134a (1,1,1,2-tetrafluoro-ethane), optionally in admixture with one or more other propellant gases, preferably selected from the group consisting of propane, butane, pentane, dimethylether, CHCIF₂, CH₂F₂, CF₃CH₃, isobutane, isopentane and neopentane.

Preferred suspensions according to this invention are those which contain as propellant gas HFA 227 on its own, a mixture of HFA 227 and HFA 134a or HFA 134a on its own. If a mixture of propellant gases HFA 227 and HFA 134a is used in the suspension formulations according to this invention, the weight ratios in which these two propellant gas components are used may be freely selected. If in the suspension formulations according to this invention one or more other propellant gases are used in addition to the propellant gases HFA 227 and/or HFA 134a, selected from the group consisting of propane, butane, pentane, dimethylether, CHCIF₂, CH₂F₂, CF₃CH₃, isobutane, isopentane and neopentane, the proportion of this other propellant gas component is preferably less than 50%, preferably less than 40% and more preferably less than 30%.

The suspensions according to this invention preferably contain between 0.001 and 0.8% tiotropium. Suspensions which contain 0.08 to 0.5%, more preferably 0.2 to 0.4% tiotropium are preferred according to this invention.

In addition, these drugs, the KMUPs and KMUPs derivative are usually administered as an aerosol with or without propellant, or as an inhaled powder, for instance with Novolizer®. This invention contemplates either co-administering both drugs in one delivery form such as an inhaler, which means putting both drugs in the same inhaler. Formulations are within the skill in the art (for instance contain excipients like lactose monohydrate).

Thus, for example, EP 0372777 requires the use of 1,1,1,2-tetrafluoroethane in combination with both a co-solvent having greater polarity than 1,1,1,2-tetrafluoroethane (e.g. an alcohol or a lower alkane) and a surfactant in order to achieve a stable formulation for a medicament powder. In particular, it is noted from the specification on Page 3, Line 7 that “it has been found that the use of propellant 134a (1,1,1,2-tetrafluoroethane) and drug as a binary mixture or in combination with a conventional surfactant such as sorbitan tri-oleate does not provide a formulation having suitable properties for use with pressurized inhalers”. Surfactants are generally recognized by those skilled in the art as essential components of an aerosol formulation, required not only to reduce aggregation of the medicament but also to lubricate the valve employed, thereby ensuring consistent reproducibility of valve actuation and accuracy of the dose dispensed. WO91/11173, WO91/11495 and WO91/14422 are concerned with formulations comprising an admixture of drug and surfactant. WO91/04011 discloses a medicinal aerosol formulation in which the particulate medicaments are pre-coated with surfactant prior to dispersal in 1,1,1,2-tetrafluoroethane.

We have now surprisingly found that, in contradistinction to these teachings, it is in fact possible to obtain satisfactory dispersions of medicaments in fluorocarbon or hydrogen-containing chlorofluorocarbon propellants such as 1,1,1,2-tetrafluoroethane without recourse to the use of any surfactant or co-solvent in the composition, or the necessity to pre-treat the medicament prior to dispersal in the propellant.

The term excipients or “pharmaceutically acceptable carrier or excipients” and “bio-available carriers or excipients” mentioned above include any appropriate compounds known to be used for preparing the dosage form, such as the solvent, the dispersing agent, the coating, the anti-bacterial or anti-fungal agent and the preservation agent or the delayed absorbent. Usually, such a carrier or excipient does not have therapeutic activity itself. Each formulation prepared by combining the derivatives disclosed in the present invention and the pharmaceutically acceptable carriers or excipients will not cause the undesired effects, allergy or other inappropriate effects when being administered to an animal or human. Accordingly, the derivatives disclosed in the present invention in combination with the pharmaceutically acceptable carrier or excipients are adaptable in clinical usage and in humans.

A therapeutic effect can be achieved by using suitable dosage forms, in part, depending upon the use or the route of administration, eg. venous, oral, and inhalation routes or via the nasal, rectal and vaginal routes. Such dosage forms should allow the compounds to reach target cells. About 0.1 mg to 1000 mg per day of the active ingredient is administered for patients with various diseases. Preferably, in one embodiment, a single oral dose of KMUPs derivative or KMUPs complex compound is about 1˜2.5 milligram per kilogram of body weight. In the treatment for respiratory conditions, the KMUPs derivative compound and KMUPs complex compound are preferably delivered by inhalation. The compound can be delivered as an aerosol, mist or powder. An effective amount for delivery by inhalation is about 2.5˜5 mM per inhalation, several times daily. The compound also can be delivered orally in amounts of about 1˜2.5 milligram per kilogram of body weight. The use of single dose oral combination therapy, the amounts of KMUPs derivative and additional active agents in a dosage form may be administered amounts of about 10˜50 milligram as a single pharmaceutical dosage composition.

The carrier varies with each formulation, and the sterile injection composition can be dissolved or suspended in non-toxic intravenous injection diluents or solvent such as 1,3-butanediol. Among these carriers, the acceptable carrier may be mannitol or water. In addition, fixing oil and synthetic glycerol ester or di-glycerol ester are commonly used solvents. A fatty acid such as oleic acid, olive oil or castor oil and glycerol ester derivatives thereof, especially the oxy-acetylated type, may serve as the oil for preparing the injection and as the natural pharmaceutically acceptable oil. Such an oil solution or suspension may include the long chain alcohol diluents or dispersing agents, carboxylmethyl cellulose or an analogous dispersing agent. Other carriers are common surfactants such as Tween and Spans or other analogous emulsion, or a pharmaceutically acceptable solid, liquid or other bio-available enhancing agent used for developing a formulation that is used in the pharmaceutical industry.

The composition for oral administration adopts any acceptable oral formulation, which includes a capsule, tablet, pill, emulsion, aqueous suspension, dispersing agent and solvent. A carrier is generally used in an oral formulation. Taking a tablet as an example, the carrier may be lactose, corn starch and lubricant, and magnesium stearate is the basic additive. The diluents used in the capsule include lactose and dried corn starch. To prepare the aqueous suspension or the emulsion formulation, the active ingredient is suspended or dissolved in an oil interface in combination with the emulsion or the suspending agent, and an appropriate amount of a sweetening agent, flavors or pigment is added as needed.

The nasal aerosol or inhalation composition may be prepared according to well-known preparation techniques. For example, the bioavailability can be increased by dissolving the composition in phosphate buffer saline and adding the benzyl alcohol or another appropriate preservative, or an absorption enhancing agent. The compound of the present invention may be formulated as a suppository for rectal or virginal administration.

The compound of the present invention can also be administered intravenously, as well as subcutaneously, parentally, muscular, or by intra-articular, intracranial, intra-articular fluid and intra-spinal injections, aortic injection, sterna injection, intra-lesion injection or other appropriate administration.

The present invention discloses a pharmaceutical composition in which the active agent is a theophylline-based moiety compound for inhibiting pulmonary artery (PA) and hypertension (PAH) in hypoxia. In particular, they are related to reduced VEGF and restored eNOS in hypoxic PAH, obstructive pulmonary disease, pulmonary artery hypertension, pulmonary artery proliferation and vascular remodeling inhibiting disease.

It is well known that the VEGF family of proteins, directly increased in hypoxia, is an important angiogenic cytokine with critical roles in angiogenesis. Recognized as the single most important angiogenic cytokine, VEGF-A has a central role in tumor biology and will likely have an important role in approaches designed to evaluate patient prognosis for pulmonary proliferative disease; it may also become an important target for an anti-proliferative agent in lung tissue through airway or oral administration.

Hypoxia is a common cause of persistent pulmonary artery hypertension in newborns and is also associated with endothelial dysfunction and abnormal pulmonary vascular remodeling. The GTPase RhoA and ROCK II have been implicated in the pathogenesis of persistent pulmonary artery hypertension, but their contribution to endothelial remodeling and function is not well known. ROCK II reportedly mediates hypoxia-induced capillary angiogenesis, a previously unrecognized but potentially important adaptive response. Sustained inhibition of the RhoA/ROCK II pathway throughout the period of hypoxic exposure attenuated pulmonary artery hypertension and prevented remodeling in blood vessels without enlarging the 100 lumen diameter.

Hypoxic pulmonary artery hypertension has been attributed to structural changes in the pulmonary vasculature, including narrowing of the vascular lumen and loss of vessels, which produces a fixed increase in resistance. Pulmonary artery hypertension is characterized by reduced eNOS, increased ROCK II expression and decreased pulmonary vascular density. eNOS downstream signaling in the cGMP-pathway for inhibiting pulmonary artery hypertension includes the expression of soluble guanylyl cyclase (sGC-α) and protein kinase G (PKG), which have been reported to be downregulated by hypoxia. In hypoxic pulmonary artery hypertension, NO release disturbances can be caused by downregulation of eNOS and upregulation of ROCK II. Vascular contractility and resistance are regulated by eNOS, sGC-α, phosphodiesterase-5A (PDE-5A) and RhoA/ROCK II expression in the cGMP-pathway. Moreover, co-localized eNOS/sGC-ca/PDE-5A expressions in a pulmonary artery are involved in sildenafil's inhibition of pulmonary artery hypertension. Although sildenafil and the ROCK inhibitors Y27632 and fasudil have been beneficial in pulmonary artery hypertension through the inactivation of RhoA/ROCK II, the relationship between the co-localized VEGF/ROCKII and treatment with buffered L-ascorbate/L-ascorbic acid remains to be investigated.

VEGF is required for the growth of pulmonary endothelial cells and is needed to repair damage caused by hypoxia. One study found pulmonary VEGF expression to be markedly decreased in an experimental model of persistent pulmonary artery hypertension, mimicking the structural and functional abnormalities of a pulmonary artery, and that exogenous treatment with VEGF improved pulmonary artery hypertension by upregulating the production of NO. The controversy between increasing and decreasing VEGF for the treatment of pulmonary artery hypertension in hypoxia remains to be resolved. VEGF exerts its biological effects primarily on endothelial cells in hypoxia. Hypoxia regulation via VEGF has been described as occurring through the induction of PI3K/Rho/ROCK and c-Myc, which is a target of the PI3K/ROCK II-signaling pathway and which can regulate a VEGF promoter through its binding element.

The expression of RhoA/ROCK II is surprisingly close to VEGF signaling. Previous reports found that RhoA or ROCK II inhibition diminishes VEGF-induced polymerization of actin. The ROCK inhibitor fasudil inhibits VEGF-induced angiogenesis in vitro and in vivo. These findings imply a close relationship between RhoA/ROCK II and VEGF. The therapeutic benefits of a ROCK inhibitor in hypoxia are partly due to the restoration of PKG-mediated vasodilatation. cGMP-dependent inhibition of Rho/ROCK II by KMUP-1-ascorbic acid complex is thus a competitive strategy to the ROCK inhibitor fasudil and cGMP-enhancer sildenafil-ascorbic acid complex for inactivating ROCK II to treat hypoxic pulmonary artery hypertension.

ROS are produced from endogenous sources, most notably the oxidative metabolism in mitochondria, and from exogenous sources such as ionizing radiation and hypoxia. ROS can induce DNA damage via hypoxia and contribute to pulmonary artery hypertension. To evaluate the protective effects of KMUP-1-ascorbic acid complex and sildenafil-ascorbic acid complex against hypoxia-induced oxidative damage, it is essential to determine ROS production during hypoxia in lung tissues, beside producing other hypoxic or normoxic organ injuries. To confirm whether KMUP-1-ascorbic acid complex and sildenafil-ascorbic acid complex can ameliorate vascular narrowing and ventricular hypertrophy, we verified our findings by observing changes in pulmonary artery wall thickness, immunohistochemistrical (IHC) staining of VEGF/eNOS involved in pulmonary artery endothelium, and hematoxylin and eosin (H&E) staining of RV/LV+S by microscopic analysis.

To prove the treatment effect on pulmonary disease from the KMUPs derivative and KMUPs complex pharmaceutical composition, we used animal hypoxia, immunohistochemistrical staining and hematoxylin-eosin staining. We confirmed that KMUPs derivative and KMUPs complex treatment inhibit pulmonary artery hypertension, via cGMP-dependent inhibition of RhoA/ROCK II (Rho kinase II) in pulmonary artery and lung tissue.

Hypoxic Animal Chamber

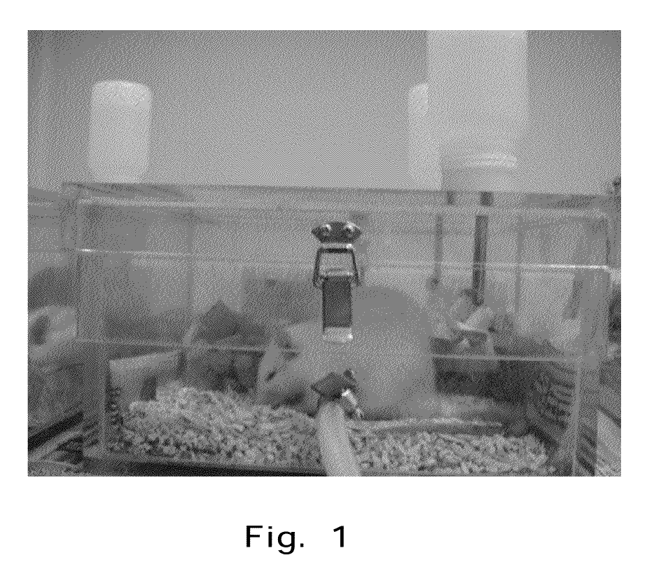

The upper panel of FIG. 1 shows the rats living in a plastic chamber under a chronic hypoxic condition, which is an animal model for experiments.

Blood pressure, heart weight and pulmonary artery hypertension

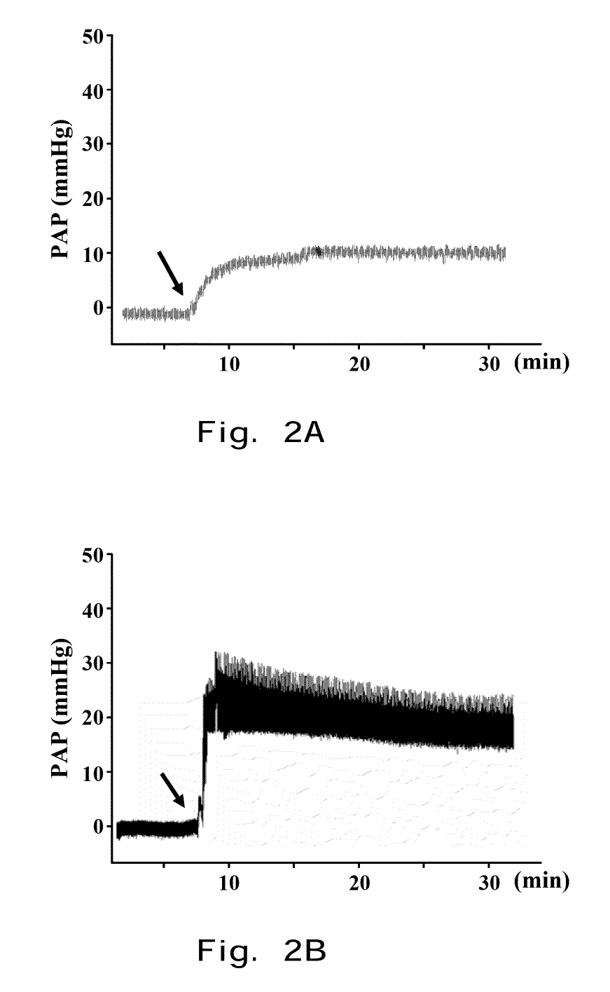

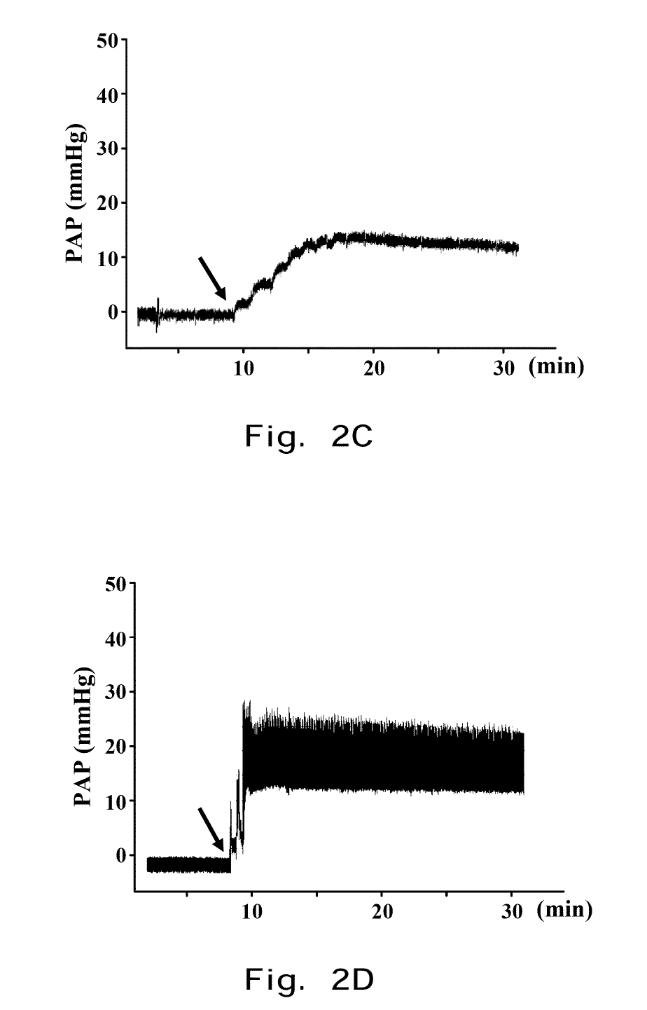

In lower panel of FIG. 2, the mean pulmonary arterial pressure (MPAP) of normoxic and hypoxic rats was 12.9±0.9 and 26.5±0.6 mmHg (n=6), respectively (FIGS. 2A, 2B). During the 21-day treatment of hypoxic rats with KMUP-1-ascorbic acid complex or sildenafil-ascorbic acid complex (5 mg kg/day, p.o.), pulmonary artery hypertension development was markedly attenuated to 16.9±1.1 and 19.8±0.7 mmHg (n=6), respectively, on the last day (FIG. 2C, 2D). Mean arterial blood pressure (MABP) and heart rate (HR) were not significantly changed by either KMUP-1-ascorbic acid complex or sildenafil-ascorbic acid complex. The heart weight/body weight ratio (HW/BW) of rats treated with KMUP-1-ascorbic acid complex or sildenafil-ascorbic acid complex was significantly different from non-treated rats after 21 days (Table 1).

TABLE 1
Effects of treatments on rat hemodynamics in hypoxia

| | | Hypoxia | | |
|---|---|---|---|---|
| | | | + | + |
| | Normoxia | | KMUP-1 A | sildenafil-A |
| BW (g) | 421.4 ± 7.7 | 237.8 ± 8.3* | 288.3 ± 7.9# | 275.0 ± 10.4# |
| HR | 375.8 ± 14.0 | 380.7 ± 12.0 | 342.1 ± 15.9 | 352.2 ± 36.4 |
| (b.p.m.) | | | | |
| MPAP | 12.9 ± 0.9 | 26.5 ± 0.6* | 16.9 ± 1.1# | 19.8 ± 0.7# |
| MPBP | 91.8 ± 1.3 | 90.4 ± 3.7 | 89.7 ± 2.9 | 93.6 ± 3.1 |
| HW/BW | 3.6 ± 0.1 | 5.1 ± 0.2* | 4.1 ± 0.3# | 4.2 ± 0.2# |

(Note)
Data were means ± SEM.;
*P < 0.05 compared with Normal;
#P < 0.05 compared with 21 day hypoxia.
KMUP-1A = KMUP-1-ascorbic acid complex (5 mg kg/day, p.o.)
sildenafil-A = sildenafil-ascorbic acid complex (5 mg kg/day, p.o.)
BW = body weight;
HR = heart rate;
b.p.m. = beats per minute
MPAP = mean pulmonary arterial pressure (mmHg),
MPBP = mean arterial blood pressure (mmHg),
HW/BW = heart weight/body weight ratio (mg/g)

H&E Staining on Lung and Heart Tissues

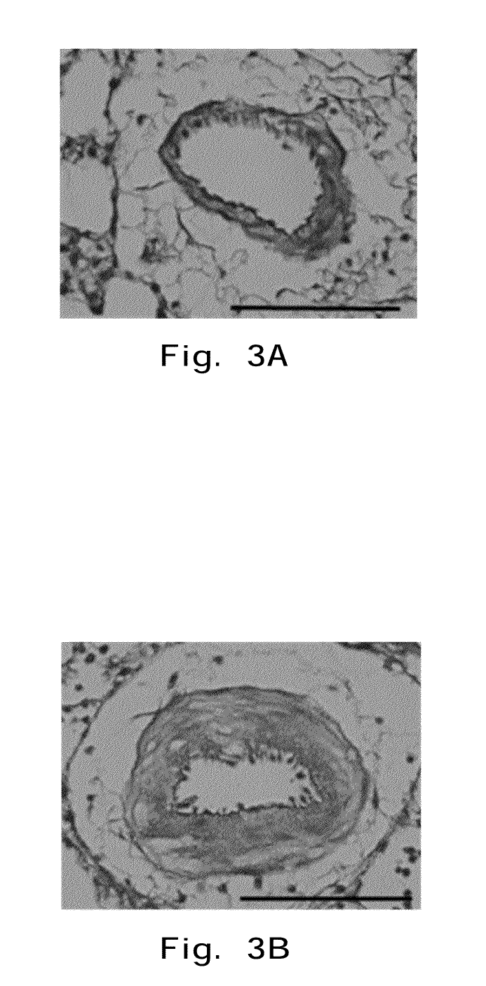

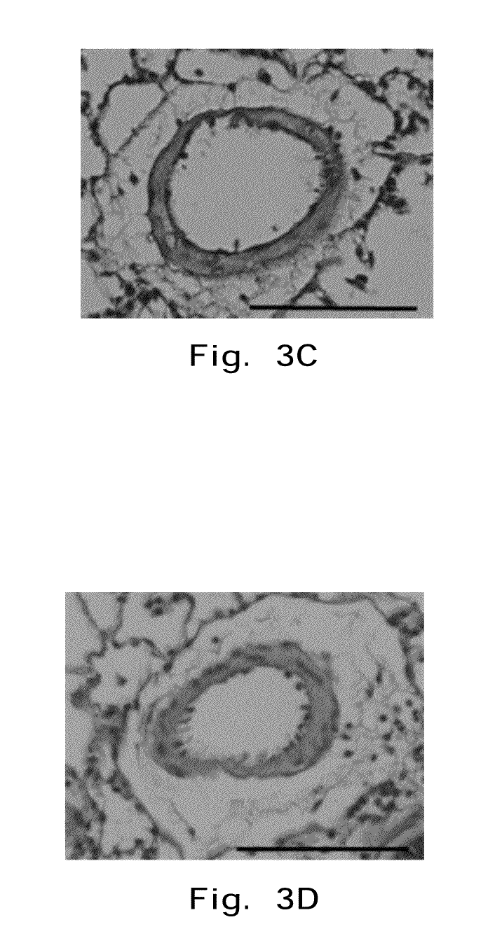

FIG. 3A indicates the cross section of a pulmonary artery. In which, vascular muscularization or remodeling is represented by an increase of pulmonary artery wall thickness (WT %) in hypoxic rats, detected on day 0 and day 21, following right lung resection. Small pulmonary arterial morphology (<150 μM) in normoxia was highly enlarged to above 180 μM diameter and reduced by KMUP-1-ascorbic acid complex in hypoxia. As shown in FIG. 3A, sections stained with H&E indicated that muscularization of the distal pulmonary artery was significantly lower in hypoxic rats treated with KMUP-1-ascorbic acid complex (FIG. 3C) and sildenafil-ascorbic acid complex (FIG. 3D) than the control group given vehicle only (FIG. 3B).

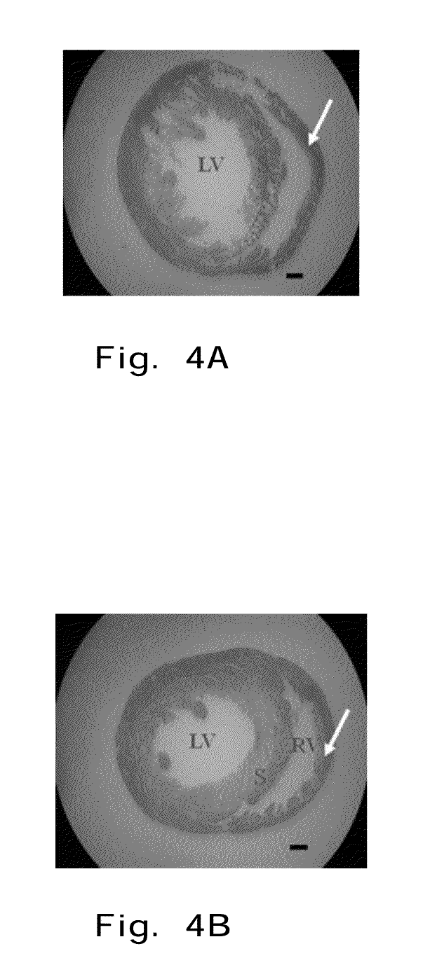

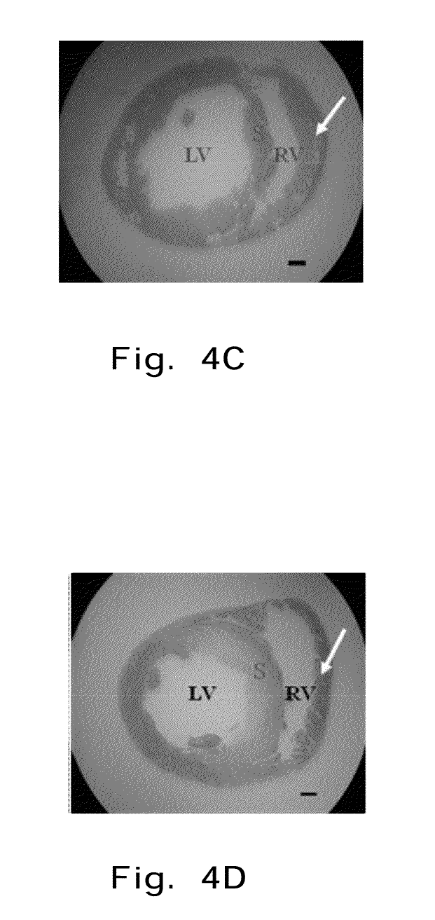

FIG. 4 indicates the cross section of a heart. FIG. 4C shows that hypoxia increased the relative right ventricle (RV)/[left ventricle (LV)+ intraventricular septum (S)] weight ratio, i.e. right heart index, to 184.6±0.7%, compared to normoxic rats (FIG. 4A). KMUP-1-ascorbic acid complex and sildenafil-ascorbic acid complex reduced the thickness of RV compared to the hypoxic stateKMUP-1-ascorbic acid complex reduced the right heart index to 117.4±2.6% (P<0.01) and sildenafil-ascorbic acid complex decreased the right heart index to 145.3±0.4% (P<0.01) (FIG. 4B, 4D).

Relative Pulmonary Artery Wall Thickness (WT %)

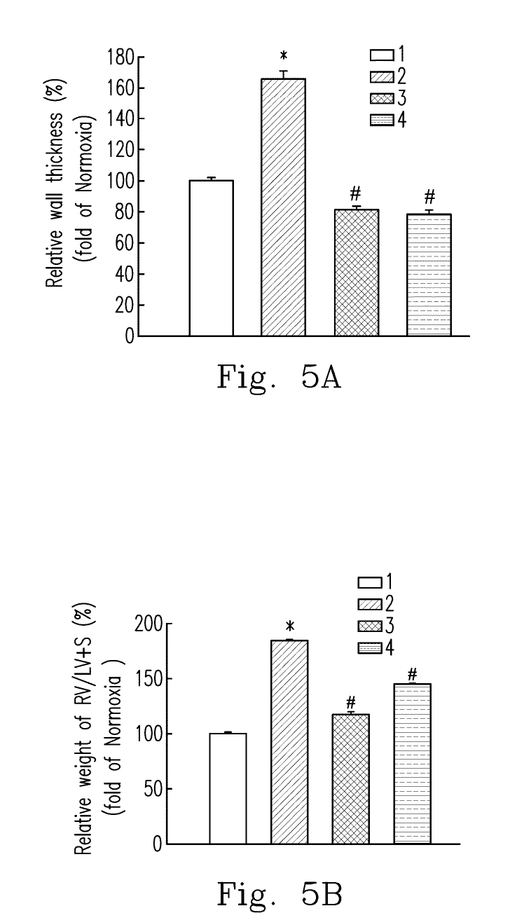

KMUP-1-ascorbic acid complex and sildenafil-ascorbic acid complex reduced the thickness of RV compared to the hypoxic state. As shown in FIG. 5B, hypoxia increased the relative right ventricle (RV)/[left ventricle (LV)+intra ventricular septum (S)] weight ratio, i.e. right heart index, to 184.6±0.7%, compared to normoxic rats. KMUP-1-ascorbic acid complex reduced the right heart index to 117.4±2.6% (P<0.01) and sildenafil-ascorbic acid complex decreased the right heart index to 145.3±0.4% (P<0.01), compared to hypoxic rats.

Immunohistochemistrical Staining of eNOS and VEGF in Pulmonary Artery Wall

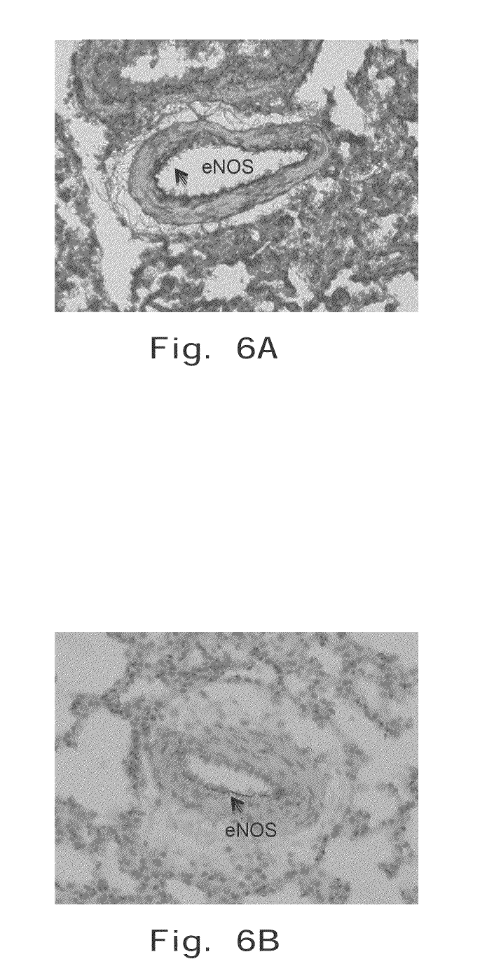

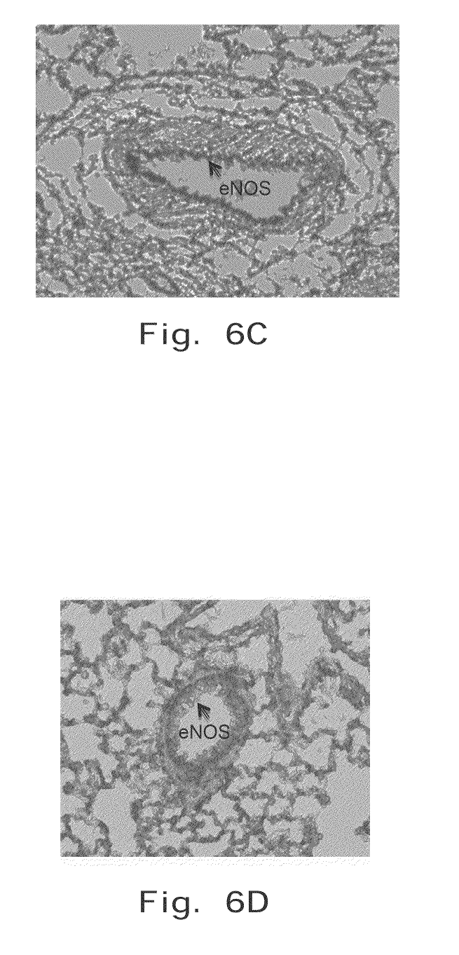

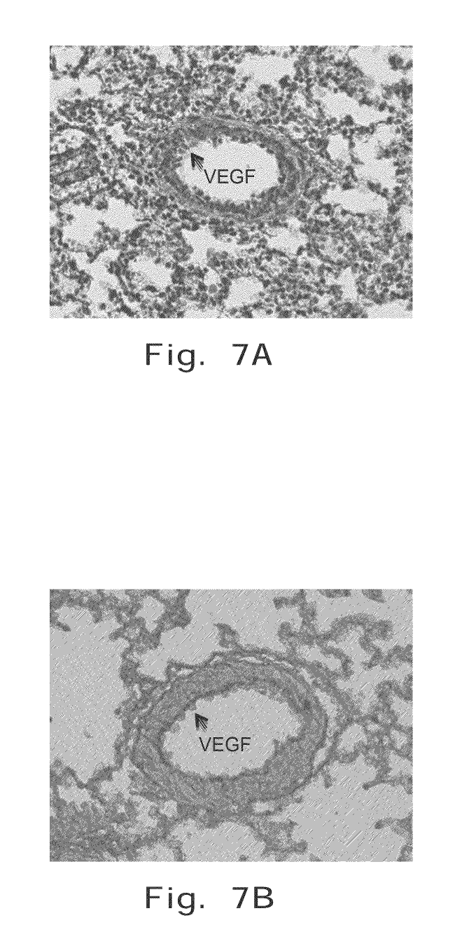

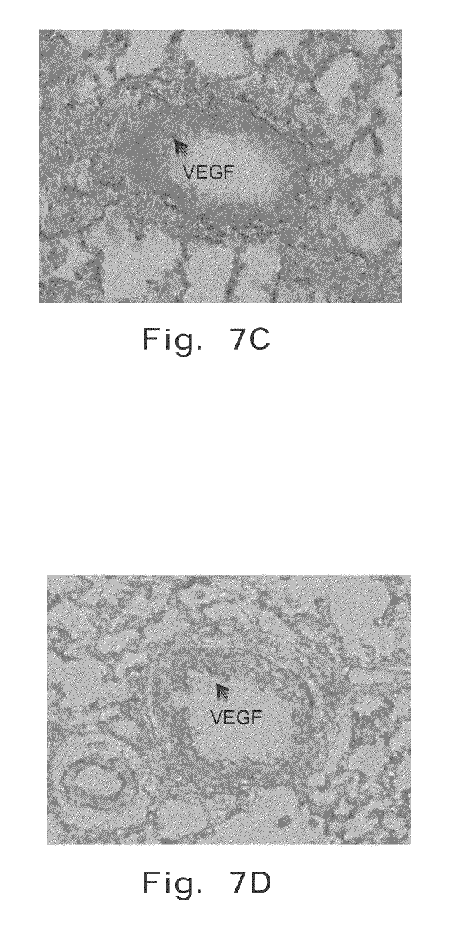

Morphometric immunostaining of lung sections of long-term hypoxic animals demonstrated a marked decrease of eNOS located mainly in pulmonary artery endothelium, and this decrease was correlated with medial thicking (FIG. 6A-6D). VEGF-immunostaining was also mainly located in the endothelium and more significantly in the smooth muscle, compared to arterial sections without immunostaining reactivity used as controls (FIG. 7A-7D).

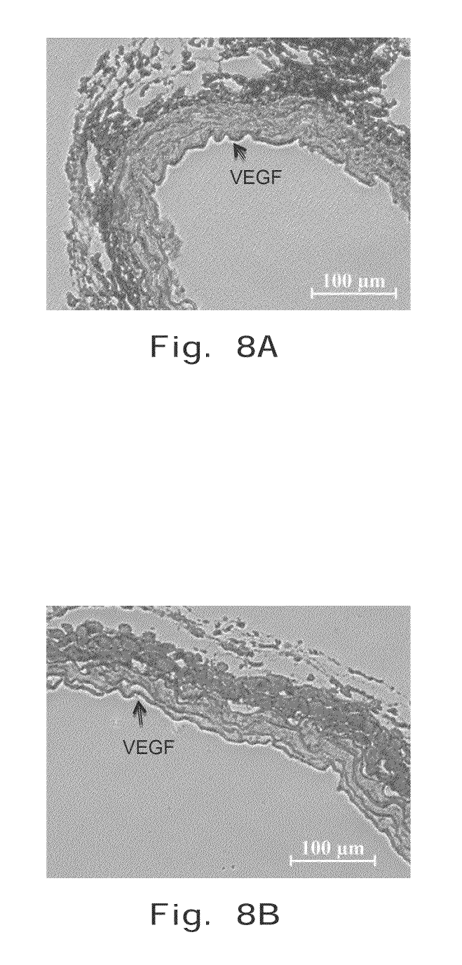

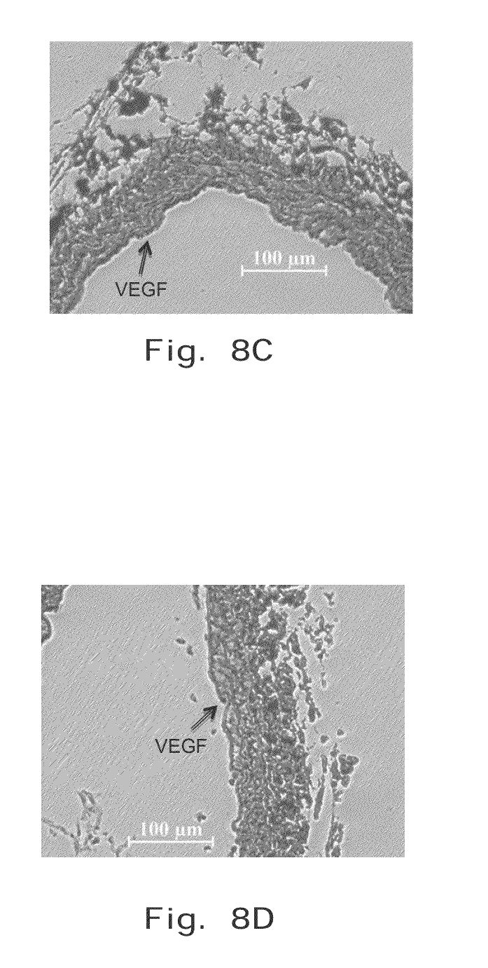

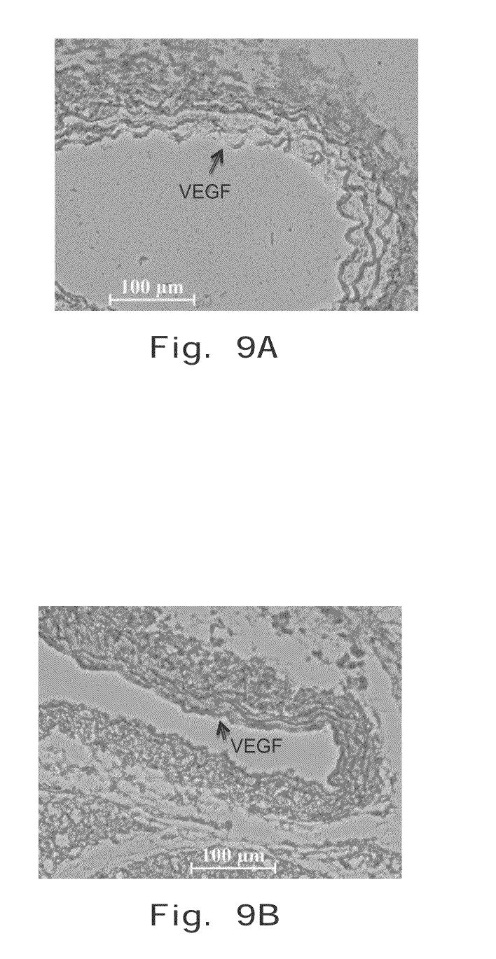

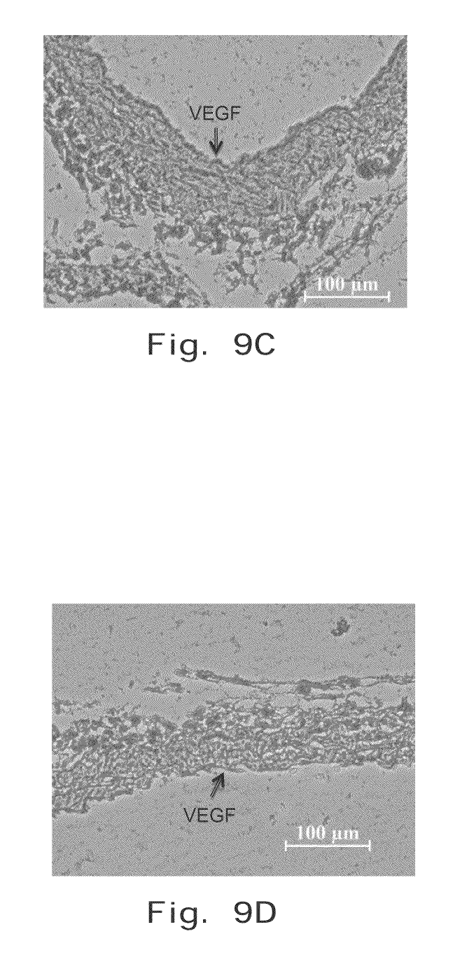

Treatments with KMUP-1-ascorbic acid complex or sildenafil-ascorbic acid complex restored the decay of eNOS and reduced VEGF immunostaining reactivity. L-ascorbic/L-ascorbate (40, 80 μM) reduced the IHC of VEGF immunostaining reactivity and restored eNOS activity in the pulmonary artery, indicating an oxidative stress defence against short-term acute hypoxia (FIG. 8C).

Pulmonary eNOS/sGC-α/PKG/PDE5A after Long-Term Hypoxia

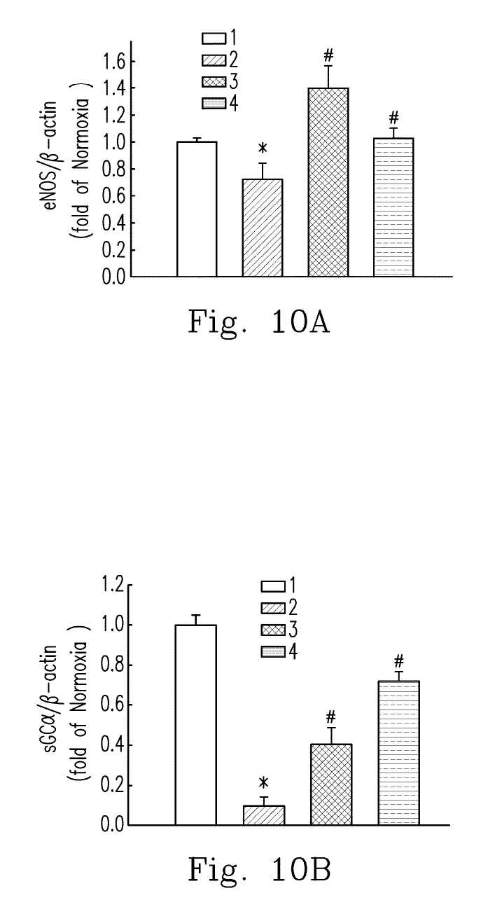

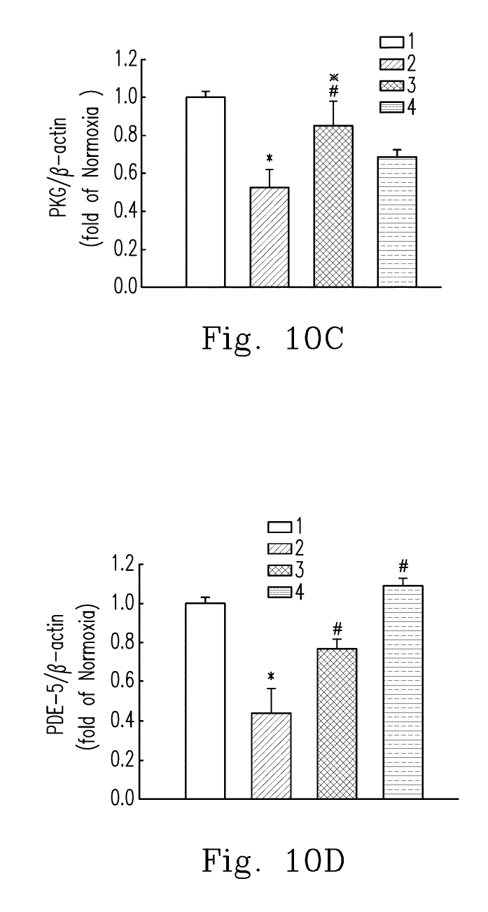

Western blotting analysis demonstrated that eNOS and sGC-α in lung tissues of hypoxia-treated rats were increased by the KMUP-1-ascorbic acid complex to 139.7±16.9% (P<0.05) and 40.4±8.4% (P<0.05) (Group 3) and by the sildenafil-ascorbic acid complex to 102.6±7.7% and 72.0±4.7% (Group 4), compared to normoxia control, respectively (FIGS. 10A and 10B). The KMUP-1-ascorbic acid complex was more potent than sildenafil-ascorbic acid complex in increasing eNOS, and sildenafil was more potent than the KMUP-1-ascorbic acid complex in increasing sGC-α expression. While KMUP-1-ascorbic acid complex clearly increased PKG to 85.0±12.9%, the sildenafil-ascorbic acid complex only brought about an insignificant increase in PKG to 68.7±3.7%, compared to non-treated hypoxic rats (FIG. 10C). PDE5A expression was decreased to 43.8±12.6% (P<0.05) in hypoxia. However, neither agent was found to further reduce the expression of PDE5A. Surprisingly, both the KMUP-1-ascorbic acid complex and sildenafil-ascorbic acid complex restored PDE5A expression to 76.9±4.9% and 109.0±3.8% of normal levels, compared to non-treated hypoxic animals (P<0.05) (FIG. 10D).

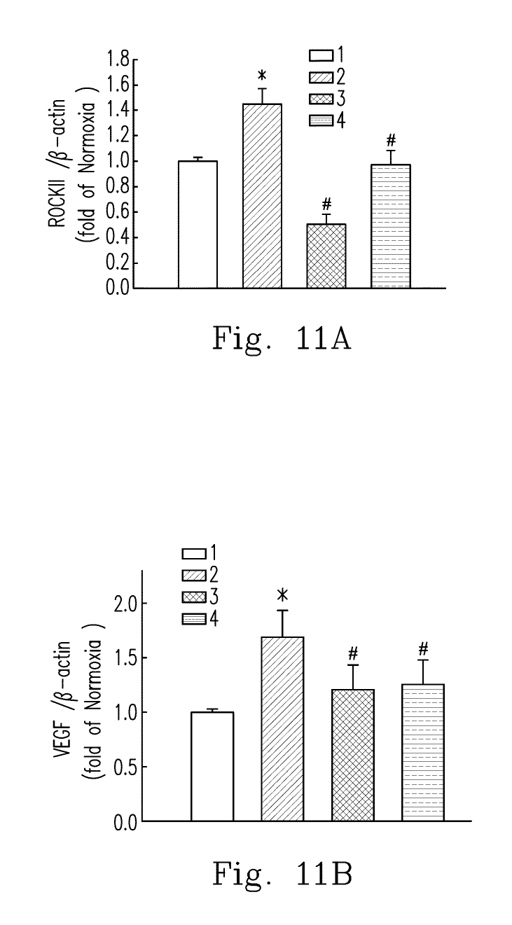

Pulmonary VEGF/ROCKII Expression after Long-Term Hypoxia Expression of ROCK II and VEGF were increased to 144.7±12.6% (P<0.05) and 168.9±24.6% (P<0.05), respectively, by 21 days of hypoxia. Treatment with KMUP-1-ascorbic acid complex or sildenafil-ascorbic acid complex during this period reduced the hypoxia-induced increase of ROCKII to 50.32±7.9% (P<0.01) and 97.4±10.9%, respectively (FIG. 11A). VEGF expression was reduced by the KMUP-1-ascorbic acid complex and sildenafil-ascorbic acid complex (120.9±22.2% and 125.7±22.5%, respectively), compared to the non-treated hypoxic rat group (FIG. 11B). KMUP-1-ascorbic acid complex was found to decrease ROCK II more potently than sildenafil-ascorbic acid complex, but these two agents were almost equally able to inhibit VEGF expression.

eNOS, ROCKII and VEGF Expression in Pulmonary Artery after Short-Term Hypoxia

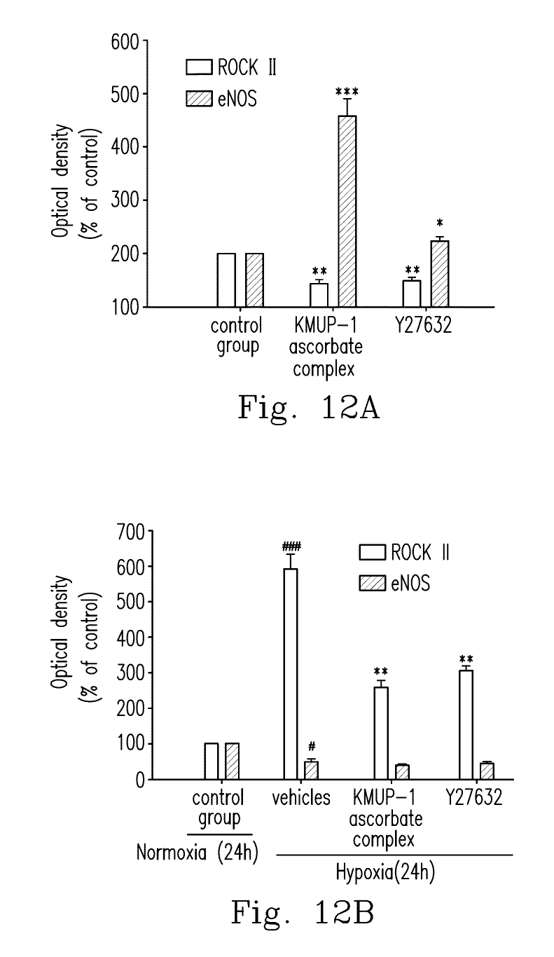

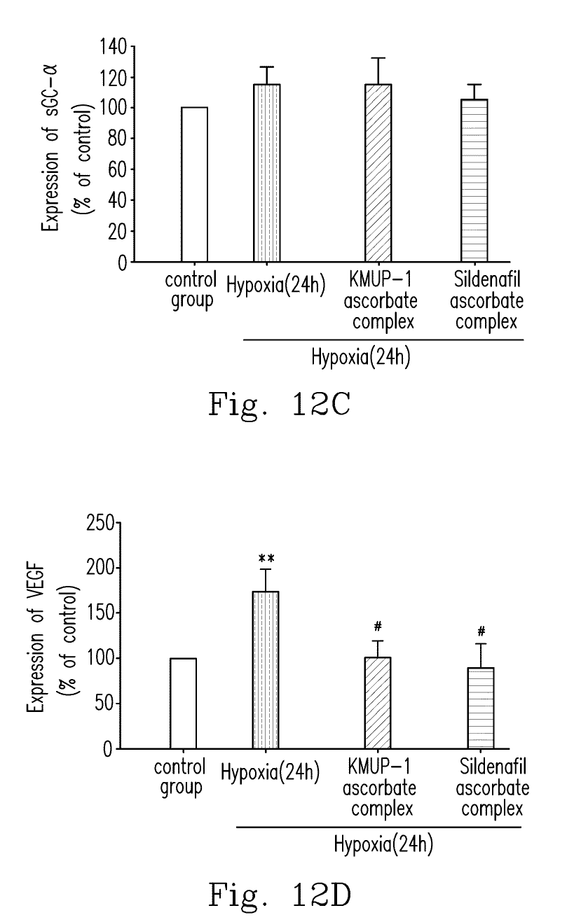

In addition to the IHC information on eNOS and VEGF above, FIG. 12A shows that the expression of eNOS in normoxic pulmonary artery rings in 24 hrs was increased to 117.9±9.1% (P<0.05) by KMUP-1-ascorbic acid complex (10 μM) and increased to 114.1±12.3% (P<0.05) by the ROCK inhibitor Y27632 (10 μM), respectively. Expression of ROCK II was decreased to 48.2±5.5% (P<0.05) by the KMUP-1-ascorbic acid complex and to 67.5±14.8% by Y27632 (P<0.01), respectively, in comparison with non-treated controls.

FIG. 12B shows that the expression of ROCK II in an isolated hypoxic pulmonary artery at 24 hrs increased to 180±10.2% (P<0.01) and eNOS % was decreased to 60±8.3% (P<0.05). Treatment with the KMUP-1-ascorbic acid complex returned ROCK II to 140±9.0% (P<0.05) and eNOS to 45±3.0%, respectively. Treatment with Y27632 returned ROCK II to 150±13.1% and eNOS to 50±4.7%, respectively, compared to normoxic control levels.

FIG. 12C shows that sGC-α showed no significant difference between hypoxia and normoxia, indicating its resistance to hypoxia. FIG. 12D shows that VEGF was increased by hypoxia but was inhibited by the KMUP-1-ascorbic acid complex and sildenafil-ascorbic acid complex, respectively.

Pulmonary NOx after Long-Term Hypoxia

Griess reagent analysis showed that the basal levels of NOx in lung tissue significantly decreased to 59.8±3.7%, compared to 100% of the control group, at 21 days of hypoxia (FIG. 13). The decreased levels of NOx in hypoxic lung tissues were significantly restored by the KMUP-1-ascorbic acid complex and sildenafil-ascorbic acid complex to 86.3±1.73 and 84.5±1.42%, respectively (##P<0.01 versus control).

Pulmonary ROS after Long-Term Hypoxia

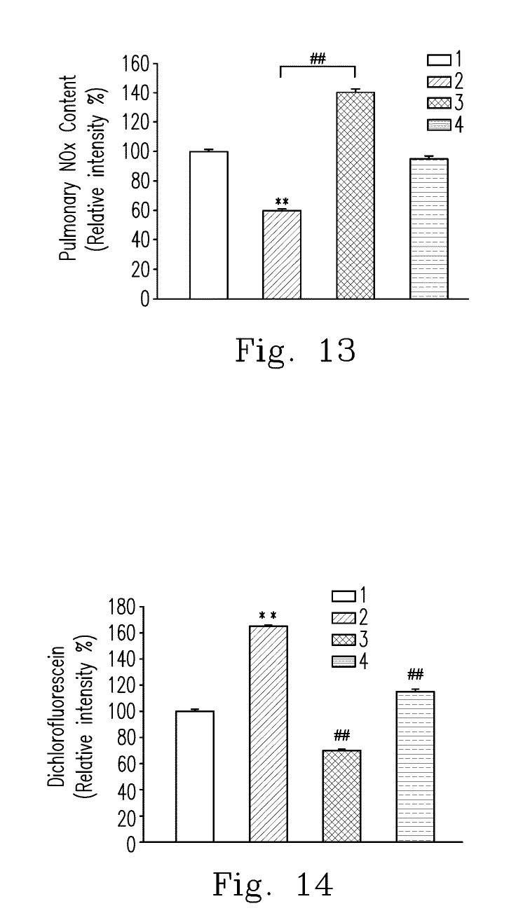

FIG. 14 shows that hypoxia increased ROS in lung tissues by 100±1.5% as detected by H2DCF-DA assay using fluorescence analysis. Hypoxia for 21 days was increased to 165±1.3%. The KMUP-1-ascorbic acid complex and sildenafil-ascorbic acid complex decreased the hypoxia-induced dichlorofluoroscence to 70±1.4% and 115±2.5%, respectively.

The histologic morphologic observations in this study confirm the benefits of L-ascorbates of KMUP-1 and sildenafil for protecting the pulmonary artery from endothelium proliferation via VEGF/eNOS, evidenced by IHC. L-ascorbic acid+L-ascorbate buffer incubated with pulmonary artery rings certainly inhibits short-term acute hypoxia-induced upregulation of VEGF and rarefaction of eNOS in IHC morphologic observation. L-ascorbic acid bound to KMUP-1 or sildenafil is thus suggested as a rational combination for improving the treatment of pulmonary artery hypertension, hypoxia-induced pulmonary endothelium hyperproliferation or oxidative stress-induced disease.

Table 2 illustrates examples of some combination therapies in the present invention wherein the combination comprises an effective amount of a KMUPs derivative compound and an additional active agent being a statins analogues, wherein said combination together comprises a pulmonary disease inhibiting effect and the amount of said compounds.

TABLE 2

| Without treatment control; | | |
|---|---|---|
| non-treatment allergic animal | iNOS | MMP-9 |
| (Vehicle) | 100 (%) | 100 (%) |
| KMUP-1 HCl (Inhal) 5 mM | 55 ± 14.8 | 42 ± 6.3 |
| KMUP-1 HCl (Inhal) 5 mM | 52 ± 13.6 | 43 ± 3.8 |
| Simvastatin (oral) 2.5 mg Kg⁻¹ | 58 ± 12.4 | 41 ± 2.5 |
| KMUP-2 HCl(Inhal) 5 mM | 47 ± 8.6 | 40 ± 3.7 |
| KMUP-2 HCl (Inhal) 5 mM | 45 ± 7.2 | 40 ± 3.7 |
| Simvastatin (oral) 2.5 mg Kg⁻¹ | | |
| KMUP-3 HCl (Inhal) 5 mM | 48 ± 7.5 | 44 ± 2.9 |
| KMUP-3 HCl (oral) 2.5 mg Kg⁻¹ | 45 ± 7.5 | 43 ± 1.6 |
| KMUP-4 HCl (Inhal) 5 mM | 35 ± 7.5 | 37 ± 2.8 |
| KMUP-4 HCl (oral) 2.5 mg Kg⁻¹ | 31 ± 6.7 | 56 ± 3.4 |

P < 0.05; significantly different from control (n = 5)
Inhal = inhalation administration
Oral = oral administration

Tables 3 and 4 illustrate examples of some combination therapies in the present invention, wherein the combination includes an effective amount of a KMUPs derivative compound and an additional active agent being a statins analogues, wherein said combination together includes the white blood cell total amounts, e.g. neutrophils, lymphocytes and eosinophils elicited in the airway lumen 24 hrs after the last ovalbumin challenge.

TABLE 3

| Without treatment control; | | | |
|---|---|---|---|
| non-treatment allergic animal | Eosinophil | Lymphocyte | Neurophil |
| (Vehicle) | 0.3 ± 0.1 | 0.7 ± 0.2 | 0.9 ± 0.2 |
| ovalbumin challenge | 37.2 ± 2.8 | 2.4 ± 0.3 | 1.0 ± 0.2 |
| KMUP-1 HCl (Inhal) 5 mM | 23.7 ± 2.8 | 1.5 ± 0.3 | 1.0 ± 0.2 |
| KMUP-5 (Inhal) 5 mM | 22 ± 2.4 | 1.3 ± 0.5 | 1.0 ± 0.3 |
| Simvastatin (oral) 2.5 mg | 27 ± 1.6 | 1.8 ± 0.7 | 1.5 ± 0.4 |
| Kg⁻¹ | | | |
| KMUP-2 HCl(Inhal) 5 mM | 25 ± 2.2 | 1.2 ± 3.7 | 1.0 ± 0.4 |
| KMUP-2 HCl (Inhal) 5 mM | 20 ± 3.5 | 1.4 ± 0.6 | 1.0 ± 0.2 |
| Simvastatin (oral) 2.5 mg | | | |
| Kg⁻¹ | | | |
| KMUP-3 HCl (Inhal) 5 mM | 24 ± 3.5 | 1.3 ± 0.6 | 1.0 ± 0.3 |
| KMUP-3 HCl (oral) 2.5 mg | 25 ± 2.5 | 1.7 ± 2.8 | 1.1 ± 0.3 |
| Kg⁻¹ | | | |
| KMUP-4 HCl (Inhal) 5 mM | 21 ± 6.7 | 1.6 ± 3.4 | 1.1 ± 0.2 |
| KMUP-4 HCl (oral) 2.5 mg | 20 ± 3.5 | 1.7 ± 1.8 | 1.1 ± 0.3 |
| Kg⁻¹ | | | |

TABLE 4

| Without treatment control; | | | |
|---|---|---|---|
| non-treatment allergic animal | Eosinophil | monocyte | Total cells |
| (Vehicle) | 0.3 ± 0.1 | 0.7 ± 0.2 | 25.6 ± 1.8 |
| ovalbumin challenge | 37.2 ± 2.8 | 2.4 ± 0.3 | 65.3 ± 3.4 |
| KMUP-1 HCl (Inhal) 5 mM | 23.7 ± 2.8 | 1.5 ± 0.3 | 50.6 ± 2.4 |
| KMUP-1S (Inhal) 5 mM | 22 ± 2.4 | 1.3 ± 0.5 | 48.2 ± 0.5 |
| Simvastatin (oral) 2.5 mgKg⁻¹ | 27 ± 1.6 | 1.8 ± 0.7 | 52.5 ± 0.4 |
| KMUP-2 HCl(Inhal) 5 mM | 25 ± 2.2 | 1.2 ± 3.7 | 51.0 ± 0.4 |
| KMUP-2 HCl (Inhal) 5 mM | 20 ± 3.5 | 1.4 ± 0.6 | 47.0 ± 0.3 |
| Simvastatin (oral) 2.5 mgKg⁻¹ | | | |
| ovalbumin challenge | 37.2 ± 2.8 | 2.4 ± 0.3 | 65.3 ± 3.4 |
| KMUP-3 HCl (Inhal) 5 mM | 24 ± 3.5 | 1.3 ± 0.6 | 52.1 ± 0.2 |
| KMUP-3 HCl (oral) 2.5 mg | 25 ± 2.5 | 1.7 ± 2.8 | 51.2 ± 0.3 |
| Kg⁻¹ | | | |
| KMUP-4 HCl (Inhal) 5 mM | 21 ± 6.7 | 1.6 ± 3.4 | 53.4 ± 0.3 |
| KMUP-4 HCl (oral) 2.5 mg | 20 ± 3.5 | 1.7 ± 1.8 | 51.8 ± 0.4 |
| Kg⁻¹ | | | |

P<0.05; significantly different from control (n=5)
Inhal=inhalation administration
Oral=oral administration
KMUP-1S=synthesized KMUP-1-Simvastatin
Compound Number×10⁴ cell/ml

In conclusion, both the KMUP-1-ascorbate complex and sildenafil-ascorbate complex prevent the development of pulmonary artery hypertension by suppressing VEGF/ROCK II and restorating eNOS in lung tissues or pulmonary arteries. Anti-oxidant buffered L-ascorbic acid could enhances the benefits of KMUP-1 and sildenafil by inhibiting hypoxia-induced oxidative stress. Inhaled KMUP-1-ascorbate complex and other KMUPs, including their powder preparation, is useful for the treatment of respiratory rehabilitation outcomes in COPD and respiratory failure in COPD, improving COPD-induced respiratory depression and COPD-induced respiratory disorder, so that the KMUPs and KMUPs derivative should be further investigated for use in human.

Biological Experiments

Animal Hypoxia

For our in vivo hypoxic experiments, 10-week old male Wistar rats were divided into four equal groups: a normoxia group without the KMUP-1-ascorbic acid complex/sildenafil ascorbic acid complex, a hypoxia group, a hypoxia+KMUP-1-ascorbic acid complex group and a hypoxia+sildenafil-ascorbic acid complex group. This study focused on the beneficial effects of L-ascorbic acid/L-ascorbate buffer, KMUP-1-ascorbic acid complex and sildenafil-ascorbic acid complex in hypoxia. All rats were maintained on a 12-h light/12-hr dark cycle at 25±1 and disclosed with food and water ad libitum. The normoxia group was housed in standard normoxic conditions and the other three treated groups were continuously housed in a hypoxic chamber (10% O₂) for 21 days, except for a 30-min interval each day when the chamber was cleaned, during which a normoxic gas mixture was prepared from compressed air. In our in vitro short-term experiment, the isolated rat pulmonary artery was grown under normoxia (20% O₂) or hypoxia (1% O₂) at 37 for 24 hrs. The heart weight and body weight ratio (HW/BW) of the rats treated with either the KMUP-1-ascorbic acid complex or sildenafil-ascorbic acid complex were measured on the 21st hypoxic day. All animal care and the experimental protocols for this study were approved by the Animal Care and Use Committee at Kaohsiung Medical University. Male Wistar rats (200-250 g) were provided by the National Laboratory Animal Breeding and Research Center (Taipei, Taiwan) and were housed under constant temperature and controlled illumination. Food and water were available ad libitum.

Hemodynamic Measurements

On the 21st day, the rats were administered with urethane (1.25 g/kg, i.p.) and their chests were opened. Their breathing was normal and their body temperature was maintained at 37. Using a pressure transducer (Gould, Model P50, U.S.A.) connected to a Pressure Processor Amplifier (Gould, Model 13-4615-52, U.S.A.), we recorded the mean pulmonary arterial pressure (MPAP) and heart rates of the rats from the femoral artery and mean artery blood pressure (MABP) from the pulmonary artery. A femoral vein was then cannulated and heparinized for intravenous administration of normal saline, KMUP-1-ascorbic acid complex, or sildenafil-ascorbic acid complex. The animals were then sacrificed by urethane overdose.

Western Blotting Analysis

Whole right lung tissues of hypoxia-treated and untreated rats were isolated, cut into small chips and placed into an extraction buffer (Tris 10 mM, pH 7.0, NaCl 140 mM, PMSF 2 mM, DTT 5 mM, NP-40 0.5%, pepstatin A 0.05 mM and leupeptin 0.2 mM) for protein extraction, and then centrifuged at 12,500 g for 30 min. To measure protein expression levels, the total proteins were extracted and Western blotting analysis was performed as described previously. Briefly, the protein extract was boiled to a ratio of 4:1 with a sample buffer (Tris 100 mM, pH 6.8, glycerol 20%, SDS 4% and bromophenol blue 0.2%). Electrophoresis was performed using 10% SDS-polyacrylamide gel (2 hr, 100 V, 40 mA, 50 mg protein per lane). Separated proteins were transferred to PVDF membranes treated with 5% fat-free milk powder to block the nonspecific IgGs (90 min, 100 V) and incubated for 1 hr with a specific protein antibody. The blot was then incubated with anti-mouse or anti-goat IgG linked to alkaline phosphatase (1:1000) for 1 hr. Immunoreactive bands were visualized using 188 horseradish peroxidase-conjugated secondary antibodies with subsequent ECL detection (GE Healthcare Bio-Sciences Corp., Piscataway, N.J., U.S.A.). Mouse or rabbit monoclonal antibody to eNOS (Upstate, NY, U.S.A.), sGCα(Sigma-Adrich, CA, U.S.A.), sGCα Santa Cruz, Calif., U.S.A.), PDE-5A (BD Tansduction, San Jose, Calif., U.S.A.), PKG (Calbiochem, San Diego, Calif., U.S.A.), ROCKII (Upstate, NY, U.S.A.), RhoA (Santa Cruze, Calif., U.S.A.) and the loading control protein β-actin (Sigma-Adrich, MO, U.S.A.) were used in our Western blot analysis.

The experiments were performed with adult male Wistar rats (300 to 350 g) in accordance with institutional guidelines after approval by the ethical review committee. Pulmonary artery hypertension development and pulmonary expression of 5-HTT were examined in rats after a 60 mg/kg single injection (i.p.) of monocrotaline (MCT). To assess the potential preventive effect of KMUP-1 on MCT-induced pulmonary artery hypertension and associated proliferation, we assigned rats at random to 2 groups of 8 animals which received KMUP-1 at 5 mg/kg/day p.o or 1 mg/kg/day i.p. All treatments were given once a day for 3 weeks after a single MCT injection (60 mg/kg i.p.) (Abe et al., 2004). On day 21, the rats were anesthetized and pulmonary artery blood pressure (MPAP) was recorded as previously described. Lung tissues were dissected for Western blotting and immunohistochemistry.

Anti-RhoA monoclonal antibody and anti-ERK1/2 rabbit antibody were purchased from Santa Cruz Biotechnology (CA, USA). Anti-eNOS and anti-ROCK (ROCKII) antibodies were purchased from Upstate Biotechnology (Lake Placid, N.Y., USA) horseradish peroxidase-conjugated polyclonal rabbit and mouse antibody were purchased from Santa Cruz Biotechnology. Anti-PKG; anti-VCAM and anti-ICAM-1 antibodies from Santa Cruz (CA, USA); anti-sGCα1, anti-MMP-9, ROCKII (a representative of Rho kinase), iNOS, IL-5, IL-13, and β-actin antibodies from Sigma Chemical Co. Goat anti-rabbit IgG horseradish peroxidase conjugated secondary antibody was purchased from Santa Cruz (CA, USA).

To examine the short-term action of treatments for hypoxia, the second branches of the main rat pulmonary artery rings were isolated and cut into 2-3 mm pieces for rapid incubation with KMUP-1-ascorbic acid complex/sildenafil-ascorbic acid complex (10 M), Y27632 (10 μM) for 24 hrs in wells of an incubation plate in a hypoxic (O₂%: 1) or normoxic (O₂%: 10) atmosphere. Western blotting analysis was performed as in lung tissue.

Histological Examination and H&E Staining

The right lobes of the rat lungs and hearts, including RV/LV+S, of six rats from each group were cut and soaked in formalin, dehydrated through graded alcohols, and embedded in paraffin wax. The lung tissue specimens fixed with formalin were embedded in paraffin, cut into 4-μm-thick sections, and subjected to H&E staining before examination by light microscope. In our histopathological study, we measured the thicking of the medial wall of the small intrapulmonary arteries under an Eclipse TE2000-S (Nikon) microscope. In heart tissues, 4-μm-thick paraffin sections were cut from paraffin-embedded tissue blocks, de-paraffinized by immersion in xylene and rehydrated. The slices were then dyed with H&E. After gentle rinsing with water, each slide was dehydrated through graded 208 alcohols and finally soaked in xylene twice.

Morphology, Immunohistochemistry (IHC) and Right Heart Index

We obtained the right lungs and hearts of six rats from each group. The samples were fixed in formalin and embedded in paraffin, and 4-μm sections were mounted onto Superfrost slides. Right lung and heart sections were stained with H&E for assessment of vascular and cardiac morphology. In each group, medial thickness (μm) and medial wall area (calculated as the area between the internal elastic lamina and the adventitia of the muscular layer of pulmonary arteries) were measured using an Eclipse TE2000-S microscope (Nikon) coupled to a color video camera (Nikon). The relative cardiac weight of right ventricle (RV)/[left ventricle (LV)+intraventricular septum (S)] ratio (i.e. right heart index) was calculated using right ventricle/left ventricle+septum (RV/LV+S). Measurements were obtained with Histolab software (Microvision Instruments, Evry, France). Six measurements of the pulmonary artery in each animal were taken and averaged. IHC of eNOS and VEGF antibody were selected for right lung section immunostaining.

IHC of eNOS and VEGF in Pulmonary Artery Walls

To make sure that ascorbic acid can protect against hypoxia-induced endothelium proliferation by anti-oxidant activity, sodium ascorbate+ascorbic acid (20, 40, 80 μM) was incubated at an equal molar ratio with the pulmonary artery rings under short-term hypoxia for 24 hrs to determine the IHC of VEGF and eNOS.

NO Metabolite (NOx) Production

On the 21st day of HFD supplementation and treatment or protection, production of NO was determined by metabolite nitrate+nitrite (NOx) in homogenized lung tissue using the Griess method. The three step Greiss test converts nitrate (NO₃⁻) into nitrite (NO₂⁻) giving a total NO₂⁻ concentration from a standard calibration curve. The lung tissues were homogenized, washed with PBS and incubated in a lysis buffer in addition to a protease inhibitor cocktail (Sigma, St. Louis, Mo.) to lung proteins. The homogenized lung tissue (100 μL) was incubated at 37 for 30 min with N-2-hydroxyethylpiperazine-N′-ethane sulphonic acid buffer (HEPES) (50 mM, pH 7.4), flavin adenine dinucleotide (5 μM), nicotinamide adenine dinucleotide phosphate (0.1 mM), distilled water (290 μl), and nitrate reductase (0.2 U/ml) to convert nitrate into nitrite. The samples were then incubated (25, 10 min) with Griess reagent [N-(1-naphthyl)-ethylenediamine: 0.2% (w/v), sulphanilamide: 2% (w/v), solubilized in double-distilled water: 95% and phosphoric acid: 5% (v/v)] and the absorbance measured at 540 nm. The levels of nitrite in the lung tissues were calibrated and compared to a sodium nitrite (0˜150 μl) standard curve. Six independent experiments were carried out and the data are reported as the mean±SEM.

ROS Production

At the 21st day of HFD supplementation and treatment or protection, pulmonary ROS was measured using 2′-7′-dichlorofluorescein (H2DCF-DA, Molecular Probe, USA). Briefly, 10 μl of homogenized lung tissue was diluted 100-fold with cold PBS and labelled with 5 μmol/L 2′-7′-dichlorofluorescein, and the mixture was incubated at 37° C. for 30 minutes followed by centrifugation 1000 rpm for 5 min. Fluorescence was measured at 485 nm excitation and 530 nm emission to determine the concentration of H₂O₂ (FLUOstar Galaxy, Germany).

eNOS- and MMP-9-Immunohistochemical Staining

The immunohistochemical analysis of eNOS enhancement by KMUP-1 was performed as follows. The lungs were perfused with saline followed by 10% formalin and then placed in 10% formalin for paraffin embedding. The lung tissues were cut into 3˜5 mm sections for slides, dewaxed in 100% xylene, and then re-hydrated in graded alcohol solutions. Throughout the procedures, slides were washed as appropriate in PBS. Antigen retrieval was performed by microwave treatment for eNOS immunostaining. Sections were treated with 3% H₂O₂ (10 min) to inhibit endogenous peroxidases and incubated with normal serum (60 min) to reduce nonspecific binding of secondary antibodies. Sections were then incubated at 4° C. overnight with eNOS antibodies. After washing off unbounded primary antibodies, sections were incubated with secondary biotinylated antibodies against mouse or rabbit (DAKO Co., Japan) (60 min), followed by incubation in avidin/biotin/horseradish peroxidase complex (DAKO Co., Japan) (30 min). Subsequently, peroxidase activity was visualized by incubation (3 min) with 0.05% 3,3′-diaminobenzidine (DAKO Co., Japan), which turns brown during a reaction. The reaction was stopped by washing with water. The slides were counterstained with hematoxylin. For immunostaining of MMP-9 in lung tissue, the de-paraffinized 3˜5 μm sections of lung tissue were performed in the above eNOS, but using an MMP-9 antibody.

Allergen-Sensitization, Challenge and Treatment

In this long-term experiment, 6-8 week old BALB/c female mice were sensitized on days 1 and 8 by intra-peritoneal injection (i.p.) of ovalbumin (10 μg) (Sigma-Aldrich, Schnelldorf, Germany) adsorbed to 2 mg aluminum hydroxide (AlumInject; Perbio, Erembodegem, Belgium). Mice were divided into three groups. In Group 1, a control group was only exposed to saline nebulization by aerosol. The other 2 groups were subjected to ovalbumin (1%) nebulization. Before each administration of ovalbumin, mice were either exposed to saline nebulization (Group 2) or KMUP-1 nebulization (5 mM) for 30 min by aerosol (Group 3).

Compounds

Anti-eNOS antibody was obtained from BD Biotechnology (New York, USA), anti-PKG antibody from Santa Cruz (CA, USA), anti-sGCα1 and β-actin antibodies and Y27632 from Sigma Chemical Co. KMUP-1 HCl was synthesized as previously described. The complex form of KMUP-1-ascorbic acid complex (7-[2-[4-(2-chlorobenzene)piperazinyl]ethyl]-1,3-dimethyl xanthine ascorbic acid) and sildenafil-ascorbic acid complex (5 mg/kg) was used throughout this study. The KMUP-1-ascorbic acid complex and sildenafil-ascorbic acid complex were synthesized by combining KMUP-1 or sildenafil base with L-ascorbic acid at an equal mole ratio. KMUP-1-ascorbic acid complex or sildenafil-ascorbic acid complex was dissolved in a vehicle (distilled water containing 0.5% methyl cellulose). Dilutions of KMUP-1-ascorbic acid complex, sildenafil-ascorbic acid complex and L-ascorbic acid/L-ascorbate (L-sodium ascorbate) were made with distilled water and then centrifuged at 1000 g for 10 min. All other reagents used were of analytical grade or higher and were obtained from commercial sources. In the in vitro test, ascorbic acid/ascorbate include L-form and DL-form salts (Aldrich-Sigma, St. Louis, Mo., USA) were considered as anti-oxidants.

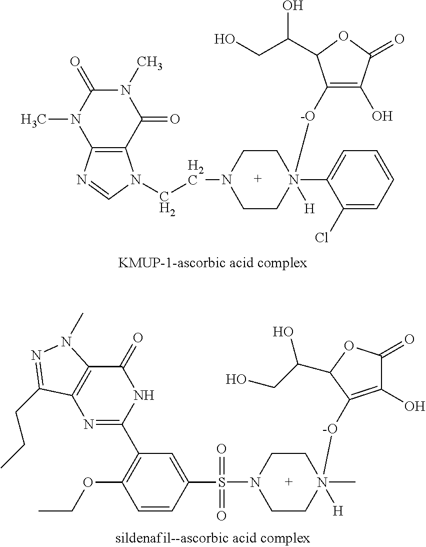

Statistical Evaluation

The results were expressed as the mean SE. Statistical differences were determined by independent and paired Student's t-test in unpaired and paired samples, respectively. Whenever a control group was compared with more than one treated group, one way ANOVA or two way repeated-measure ANOVA was used. When the ANOVA showed a statistical difference, Dunnett's or Student-Newman-Keuls test was applied. A P value less than 0.05 was considered significant in all experiments. Analysis of the data and plotting of the figures were done using SigmaPlot software (Version 8.0, Chicago, Ill., U.S.A.) and SigmaStat (Version 2.03, Chicago, Ill., U.S.A.) run on an IBM compatible computer.

### Example 1

### Preparation of KMUP-1 HCl salt (7-[2-[4-(2-chlorophenyl)piperazinyl]ethyl]-1,3-dimethylxanthine HCl)

KMUP-1 (8.0 g) was dissolved in a mixture of ethanol (10 mL) and 1N HCl (60 mL) and reacted at 50° C. for 10 min. The methanol was added to the solution under room temperature and the solution was incubated overnight for crystallization. The crystals were filtrated to obtain the precipitate of KMUP-1 HCl salt (7.4 g).

### Example 2

### Preparation of KMUP-3 HCl salt (7-[2-[4-(4-nitrobenzene)piperazinyl]ethyl]-1,3-dimethyl xanthine HCl)

KMUP-3 (8.3 g) was dissolved in a mixture of ethanol (10 mL) and 1N HCl (60 mL). The solution was reacted at 50° C. for 20 min, the methanol was added thereto under room temperature, and the solution was incubated overnight for crystallization and filtrated to obtain KMUP-3 HCl salt (6.4 g).

### Example 3

### Preparation of KMUP-1-γ-Polyglutamic Acid Complex

Method 1: Sodium γ-polyglutamic acid (20 g) was suspended in distilled water and added to KMUP-1 HCl (16 g) dissolved in 100 ml of methanol to reflux in a three-neck reactor, equipped with a condenser, for 1 hour. After cooling, obtained precipitate was dissolved in 100 ml of methanol and the resulting solution was incubated for crystallization and filtrated to obtain KMUP-1-γ-Polyglutamic acid complex (35.6 g).

Method 2: KMUP-1 HCl (16 g) is dissolved in 100 ml of methanol and added with sodium alginic acid 20 g dissolved in 100 ml of methanol to reflux in a three-neck reactor, equipped with a condenser, for 1 hour. After cooling, obtained precipitate was filtrated and re-crystallized with 100 ml of methanol to obtain the KMUP-1-γ-Polyglutamic acid complex (35.8 g).

### Example 4

### Preparation of KMUP-3-Nicotinic Acid Complex

KMUP-3 (8.3 g) was dissolved in a mixture of ethanol (10 mL) and Nicotinic acid (2.4 g). The solution was reacted at 50° C. for 20 min, the methanol is added thereto under room temperature, and the solution was incubated overnight for crystallization and filtrated to obtain the KMUP-3-Nicotinic acid complex (8.3 g).

### Example 5

### Preparation of KMUP-1-Simvastatinic Complex

KMUP-1 (8.0 g) was dissolved in a mixture of ethanol (10 mL) and HCl (1 N, 60 mL) and reacted at 50° C. for 10 min, the methanol was added thereto under room temperature, and the solution was incubated overnight for crystallization and filtrated to obtain KMUP-1 HCl (7.4 g). KMUP-1 HCl salt (4.4 g) was then redissolved in ethanol (150 mL) for use.

In a flask equipped with a magnetic stirrer, simvastatin (4.2 g) was dissolved in a mixture of ethanol (50 mL), an aqueous solution of sodium hydroxide (4 g/60 ml) and the above-mentioned filtrate of KMUP-1 HCl salt reacted with the ethanol were added then to react under room temperature. The mixture was reacted at 50° C. for 20 mins, rapidly filtrated and incubated one hour for crystallization to obtain the KMUP-1-Simastatinic complex.

### Example 6

### Preparation of KMUP-2-Polyacrylic Acid Complex

KMUP-2 HCl (8 g) was dissolved in a mixture of methanol (100 mL) and sodium polyacrylic acid (2.5 g). The solution was refluxed in a three-neck reactor, equipped with a condenser, for 1 hour. After cooling, obtained precipitate was re-dissolved in 100 ml of methanol, and the resulting solution was incubated for crystallization and filtrated to obtain the KMUP-2-polyacrylic acid complex (7.4 g).

### Example 7

### Preparation of KMUP-1-Ascorbate Complex from KMUP-1 Base and L-Ascorbic Acid

In a flask equipped with a magnetic stirrer, KMUP-1 base (13.2 g) was dissolved in a mixture of ethanol (100 mL) and an ethanol solution of equal mole ascorbic acid was then added to react at 50 for 20 mins. After cooling, a white precipitate was obtained and the sodium chloride was removed by filtration. The solvent methanol (100 mL) was added to resolve the precipitate under room temperature and incubated overnight for re-crystallization. The KMUP-1-ascorbate complex compound (16.8 g) was obtained after filtering the crystals.

### Example 8

### Preparation of KMUP-2 Oleate Complex

Method 1: 8.5 g of sodium oleic acid was suspended in distilled water and added to 12.1 g of KMUP-2 HCl dissolved in 100 ml of methanol to reflux in a three-neck reactor, equipped with a condenser, for 1 hour. After cooling, the obtained precipitate was dissolved in 100 ml of methanol and the resulting solution was incubated for crystallization and filtrated to obtain the KMUP-2 oleate complex (16.3 g).

### Example 9

### Preparation of KMUP-3 ascorbate complex

KMUP-3 HCl (8.5 g) was dissolved in 100 ml of ethanol and added to 3.9 g of sodium ascorbic acid and refluxed in a three-neck reactor, equipped with a condenser, for 1 hour. After cooling, the obtained precipitate was filtrated and re-crystallized with 100 ml of methanol to obtain the KMUP-3 ascorbate complex (9.7 g).

### Example 10

### Preparation of KMUP-3 CMC complex

5.3 g of sodium CMC was dissolved in water to form a 5% viscous aqueous solution (40 ml). KMUP-3 HCl (9.2 g) was added to the solution and the mixture was stirred at 50° C. for 1 hr to obtain a white precipitate. The solution was poured out, and then the ethanol (100 ml) was added for dehydration. Ethanol (100 ml) was additionally added to wash out the unreacted KMUP-3, overnight and warmed in a water bath at 50° C. and cooled to room temperature overnight to obtain the precipitate of the KMUP-3 CMC complex (11.2 g).

### Example 11

### Preparation of the Composition in Tablets

Tablets were prepared using standard mixing and formulation techniques as described in U.S. Pat. No. 5,358,941, to Bechard et al., issued Oct. 25, 1994, which is incorporated by reference herein in its entirety.

| | |
|---|---|
| KMUP-3 Citric acid complex | 100 mg |
| Lactose | qs |
| Corn starch | qs |

### Example 12

### Preparation of the Composition in Tablets

Tablets were prepared using standard mixing and formulation techniques as described in U.S. Pat. No. 5,358,941, to Bechard et al., issued Oct. 25, 1994, which is incorporated by reference herein in its entirety.

| | |
|---|---|
| Simvastatin | 50 mg |
| Lactose | qs |
| Corn starch | qs |

### Example 13

### Preparation of the Liquid Mist or Dry Powder Inhaler

Aerosol were prepared using standard mixing and formulation techniques as described in U.S. Pat. No. 6,713,047, to David et al., issued Mar. 30, 2004, which is incorporated by reference herein in its entirety. Each inhalation contains 2˜4 mg of the KMUP-1-ascorbate or 2˜4 mg of Sildenafil-ascorbate which can be used for 50˜75 inhalations, i.e. 200˜400 mg as the total amount, administered by a b.i.d., a t.i.d. and a q.i.d. respectively during a period of 25 days, from a dry powder or liquid mist inhaler, stored below 30° C. Powder inhalations were prepared using formulation techniques as described in U.S. Pat. No. 8,097,605, to Maus et al., issued Jan. 17, 2012, which is incorporated by reference herein in its entirety.

| | |
|---|---|
| KMUP-1 base | 200 mg |
| Ascorbic acid | QS (to adjust pH) |
| Glycerol | 1.3% (w/w) |
| HFA | to 100 mL |

b.i.d.
for 50 inhalations within 25 days in a liquid mist inhaler stored below 30° C.

Formulation II:

| | |
|---|---|
| KMUP-1 base | 300 mg |
| Ascorbic acid | QS (to adjust pH) |
| Glycerol | 1.3% (w/w) |
| HFA | to 100 mL |

t.i.d.
for 75 inhalations within 25 days in a liquid mist inhaler stored below 30° C.

Formulation III:

| | |
|---|---|
| Nanometric KMUP-1-Ascorbate | 200 mg |
| Nanometric Ascorbic acid | QS (to adjust pH) |
| Nanometric Lactose | 100 mg |

b.i.d
for 50 inhalations within 25 days in a dry powder inhaler stored below 30° C.

Formulation IV:

| | |
|---|---|
| Nanometric KMUP-1-Ascorbate | 300 mg |
| Nanometric Ascorbic acid | QS (to adjust pH) |

t.i.d
for 75 inhalations within 25 days in a dry powder inhaler stored below 30° C.

Formulation V:

| | |
|---|---|
| KMUP-1 HCl | 200 mg |
| Ascorbic acid | QS (to adjust pH) |
| Glycerol | 1.3% (w/w) |
| HFA | to 100 mL |

b.i.d.
for 50 inhalations within 25 days in a liquid mist inhaler stored below 30° C.

Formulation VI:

| | |
|---|---|
| Nanometric KMUP-1 HCl | 300 mg |
| Nanometric Ascorbic acid | QS (to adjust pH) |
| Nanometric Lactose | 150 mg |

t.i.d.
for 75 inhalations within 25 days in a dry powder inhaler stored below 30° C.

Formulation VII:

| | |
|---|---|
| Nanometric KMUP-1-Citrate | 200 mg |
| Nanometric Ascorbic acid | QS (to adjust pH) |
| Nanometric Lactose | 100 mg |

b.i.d.
for 50 inhalations within 25 day in a dry powder inhaler stored below 30° C.

Formulation VIII:

| | |
|---|---|
| KMUP-1-Citrate | 300 mg |
| Glycerol | 1.3% (w/w) |
| Ascorbic acid | QS (to adjust pH) |
| HFA | 100 mL |

t.i.d.
for 75 inhalations within 25 days in a liquid mist inhaler stored below 30° C.

Formulation IX:

| | |
|---|---|
| KMUP-1 HCl | 200 mg |
| Glycerol | 1.3% (w/w) |
| Ascorbic acid | QS (to adjust pH) |
| HFA | to 100 mL |

b.i.d.
for 50 inhalations within 25 days in a liquid mist inhaler stored below 30° C.

Formulation X:

| | |
|---|---|
| KMUP-1 HCl | 300 mg |
| Glycerol | 1.3% (w/w) |
| Ascorbic acid | QS (to adjust pH) |
| HFA | to 100 mL |

t.i.d.
for 75 inhalations within 25 days in a liquid mist inhaler stored below 30° C.

Formulation XI:

| | |
|---|---|
| Nanometric Sildenafil-Ascorbate | 200 mg |
| Nanometric Lactose | 100 mg |

t.i.d.
for 75 inhalations within 25 days in a dry powder inhaler stored below 30° C.

Formulation XII:

| | |
|---|---|
| Nanometric Sildenafil-Ascorbate | 300 mg |
| Nanometric Lactose | 150 mg |

t.i.d.
for 75 inhalations within 25 days in a dry powder inhaler stored below 30° C.

Formulation XIII:

| | |
|---|---|
| Sildenafil-Citrate | 200 mg |
| Glycerol | 1.3% (w/w) |
| HFA | to 100 mL |

b.i.d.
for 50 inhalations within 25 days in a liquid mist inhaler stored below 30° C.

Formulation XIV:

| | |
|---|---|
| Sildenafil-Citrate | 300 mg |
| Glycerol | 1.3% (w/w) |
| HFA | to 100 mL |

t.i.d.
for 75 inhalations within 25 days in a liquid mist inhaler stored below 30° C.

Formulation XV:

| | |
|---|---|
| Sildenafil base | 200 mg |
| Glycerol | 1.3% (w/w) |
| HFA | to 100 mL |
| Ascorbic acid | QS (to adjust pH) |

b.i.d.
for 50 inhalations within 25 days in a liquid mist inhaler stored below 30° C.

Formulation XVI:

| | |
|---|---|
| Nanometric Sildenafil base | 300 mg |
| Nanometric Lactose | 150 mg |
| Nanometric Ascorbic acid | QS (to adjust pH) |

t.i.d.
for 75 inhalations within 25 days in a dry powder inhaler stored below 30° C.

Formulation XVII:

| | |
|---|---|
| Nanometric KMUP-1-ascorbate | 400 mg |
| Nanometric Lactose | 200 mg |
| Nanometric Ascorbic acid | QS (to adjust pH) |

q.i.d.
for 100 inhalations within 25 days in a dry powder inhaler stored below 30° C.

Formulation XVIII:

| | |
|---|---|
| Nanometric KMUP-1 base | 400 mg |
| Nanometric Lactose | 200 mg |
| Nanometric Ascorbic acid | QS (to adjust pH) |

q.i.d.
for 100 inhalations within 25 days in a dry powder inhaler stored below 30° C.

Formulation XIX:

| | |
|---|---|
| Nanometric sildenafil ascorbate | 400 mg |
| Nanometric Lactose | 200 mg |
| Nanometric Ascorbic acid | QS (to adjust pH) |

q.i.d.
for 100 inhalations within 25 days in a dry powder inhaler stored below 30° C.

Formulation XX:

| | |
|---|---|
| Nanometric Sildenafil base | 400 mg |
| Nanometric Lactose | 150 mg |
| Nanometric Ascorbic acid | QS (to adjust pH) |

q.i.d.
for 100 inhalations within 25 days in a dry powder inhaler stored below 30° C.
   Note: one inhaled preparation includes 4.0 mg of powder (including nanometric powder) or 4 ml of an inhaled liquid preparation of a KMUPs derivative, Sildenafil Analogs derivative compound or their base (non-salt form) for use. Thus, one inhaler contains the dosage of 200 mg for 50 inhalations during 25 days, i.e. 4 mg per inhalation×2 times (b.i.d)×25 days=200 mg, or 4 mg/one inhalation×3 times (t.i.d)×25 days=300 mg, or i.e. 4 mg/one inhalation×4 times (q.i.d.)×25 days=400 mg.

### Embodiments

1. A method for inhibiting a pulmonary inflammation, comprising steps of: providing a subject suffering from the pulmonary inflammation; and administering one of a KMUPs complex compound represented by one of formula II and formula III, and a pharmaceutical composition thereof to the subject in a dosage between 1 and 5.0 milligrams per kilogram of body weight,

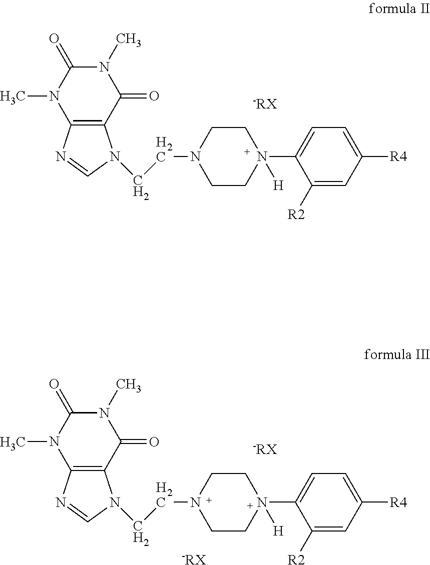

wherein: R2 and R4 are each selected independently from a group consisting of a C1˜C5 alkoxy group, a hydrogen, a nitro group, and a halogen atom;
RX includes a carboxylic group selected from a group consisting of a Statin analogue, a Co-polymer, a poly-γ-polyglutamic acid derivative, ascorbic acid, an oleic acid, a phosphoric acid, a citric acid, a nicotinic acid and a sodium CMC.
2. The pharmaceutical composition of Embodiment 1, wherein the halogen atom is one selected from a group consisting of a fluorine, a chlorine, a bromine and an iodine.
3. The pharmaceutical composition of any one of Embodiments 1-2, wherein the compound of formula I is a KMUPS derivative compound.
4. The pharmaceutical composition of any one of Embodiments 1-3, wherein the KMUPs derivative compound is one selected from a group consisting of KMUP-1, KMUP-2, KMUP-3, KMUP-4 and its pharmaceutical acceptable salts.
5. A method of providing a medical effect for inhibiting pulmonary inflammation, comprising steps of providing a subject in need thereof; and administering an effective amount of pharmaceutical composition of a KMUPs derivative to the subject in need thereof.
6. The method of Embodiment 5, wherein the KMUPs derivative compounds is one selected from a group consisting of KMUP-1, KMUP-2, KMUP-3 and KMUP-4.
7. The method of any one of Embodiments 5-6, wherein the administration is performed by one selected from an oral, injection, inhalation and topical administration.
8. The method for inhibiting pulmonary inflammation of any one of Embodiments 5-7, comprising a step of: combination administrating a pharmaceutically effective amount of a compound of KMUPs derivative and a statins analogues to the subject in need thereof.
9. The method of any one of Embodiments 5-8, wherein the statins analogues is one selected from a group consisting of Atorvastatin, Cerivastatin, Fluvastatin, Lovastatin, Mevastatin, Pravastatin, Rosuvastatin, Simvastatin and Pramastatinic acid.
10. An obstructive pulmonary disease inhibiting pharmaceutical composition including an effective amount of a compound of 7-[2-[4-(2-chlorobenzene)piperazinyl]ethyl]-1,3-dimethyl xanthine-ascorbic acid complex; and a pharmaceutically acceptable carrier.
11. An obstructive pulmonary disease inhibiting pharmaceutical composition including an effective amount of a compound of 7-[2-[4-(2-chlorobenzene)piperazinyl]ethyl]-1,3-dimethyl xanthine-ascorbic acid complex; and a pharmaceutically acceptable carrier.
12. An obstructive pulmonary disease inhibiting pharmaceutical composition including an effective amount of a compound of 7-[2-[4-(2-methoxybenzene)piperazinyl]ethyl]-1,3-dimethyl xanthine-ascorbic acid complex; and a pharmaceutically acceptable carrier.
13. An obstructive pulmonary disease inhibiting pharmaceutical composition including an effective amount of a compound of 7-[2-[4-(2-methoxybenzene)piperazinyl]ethyl]-1,3-dimethyl xanthine-ascorbic acid complex; and a pharmaceutically acceptable carrier.
14. An obstructive pulmonary disease inhibiting pharmaceutical composition including an effective amount of a compound of 7-[2-[4-(2-methoxybenzene)piperazinyl]ethyl]-1,3-dimethyl xanthine-ascorbic acid complex; and a pharmaceutically acceptable carrier.
15. An obstructive pulmonary disease inhibiting pharmaceutical composition including an effective amount of a compound of 7-[2-[4-(4-nitrobenzene)piperazinyl]ethyl]-1,3-dimethyl xanthine-ascorbic acid complex; and a pharmaceutically acceptable carrier.
16. An obstructive pulmonary disease inhibiting pharmaceutical composition including an effective amount of a compound of 7-[2-[4-(4-nitrobenzene)piperazinyl]ethyl]-1,3-dimethyl xanthine-ascorbic acid complex; and a pharmaceutically acceptable carrier.
17. An obstructive pulmonary disease inhibiting pharmaceutical composition including an effective amount of a compound of 7-[2-[4-(4-nitrobenzene)piperazinyl]ethyl]-1,3-dimethyl xanthine-ascorbic acid complex; and a pharmaceutically acceptable carrier.
18. An obstructive pulmonary disease inhibiting pharmaceutical composition including an effective amount of a compound of 7-[2-[4-(2-nitrobenzene)piperazinyl]ethyl]-1,3-dimethyl xanthine-ascorbic acid complex; and a pharmaceutically acceptable carrier.
19. An obstructive pulmonary disease inhibiting pharmaceutical composition including an effective amount of a compound of 7-[2-[4-(2-nitrobenzene)piperazinyl]ethyl]-1,3-dimethyl xanthine-ascorbic acid complex; and a pharmaceutically acceptable carrier.
20. An obstructive pulmonary disease inhibiting pharmaceutical composition including an effective amount of a compound of 7-[2-[4-(2-nitrobenzene)piperazinyl]ethyl]-1,3-dimethyl xanthine-ascorbic acid complex; and a pharmaceutically acceptable carrier.
21. An anti-pulmonary artery hypertension pharmaceutical composition including:

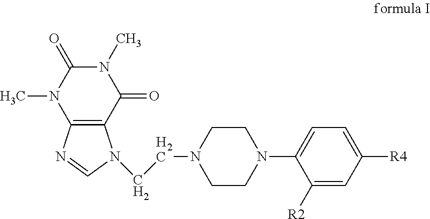

an effective amount of a compound of formula I, wherein R₂ and R₄ are each selected independently from the group consisting of a C1˜C5 alkoxy group, a hydrogen, a nitro group, and a halogen atom; and a pharmaceutically acceptable carrier.
22. The pharmaceutical composition of Embodiment 21, wherein the halogen atom is one selected from a group consisting of a fluorine, a chlorine, a bromine and an iodine.
23. The pharmaceutical composition of any one of Embodiments 21-22, wherein the compound of formula I is a KMUPs derivative compound.
24. The pharmaceutical composition of any one of Embodiments 21-23, wherein the KMUPs derivative compound is one selected from a group consisting of KMUP-1, KMUP-2, KMUP-3, KMUP-4 and its pharmaceutical acceptable salts.
25. The pharmaceutical composition of Embodiments 21-24, wherein the compound is a treatment for acute or chronic pulmonary artery hypertension.
26. A combination therapy method of providing a medical effect for the inhibition of pulmonary inflammation, comprising steps of providing a subject in need thereof; and administering an effective amount of a pharmaceutical composition of a KMUPs derivative and a statins analogues to the subject in need thereof.
27. A method of Embodiment 26, wherein the pharmaceutical composition comprising:

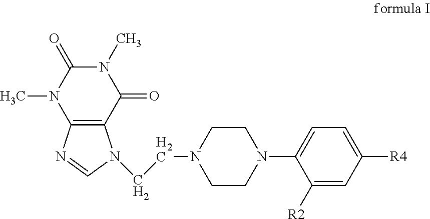

an effective amount of a KMUP derivative compound of formula I, wherein R₂ and R₄ are each selected independently from the group consisting of a C1˜C5 alkoxy group, a hydrogen; a nitro group, and a halogen atom; and a pharmaceutically acceptable carrier.
28. A method of any one of Embodiments 26-27, wherein the halogen atom is one selected from a group consisting of a fluorine, a chlorine, a bromine and an iodine.
29. A method of any one of Embodiments 26-28, wherein the pharmaceutical composition of KMUPs derivative compounds is one selected from a group consisting of KMUP-1, KMUP-2, KMUP-3, KMUP-4 and its pharmaceutical acceptable salts.
30. The method of any one of Embodiments 26-29, wherein the pharmaceutical composition of statins analogues is one selected from a group consisting of Atorvastatin, Cerivastatin, Fluvastatin, Lovastatin, Mevastatin, Pravastatin, Rosuvastatin, Simvastatin, Pramastatinic acid and its pharmaceutical acceptable salts.
31. A combination therapy method of providing a medical effect for the prevention of allergic pulmonary vascular inflammation, comprising steps of providing a subject in need thereof; and administering an effective amount of a pharmaceutical composition of a Piperazinyl Analogs compound and a statins analogues to the subject in need thereof.
32. A combination therapy method of Embodiment 31, wherein the Piperazinyl Analogs compounds is one selected from a group consisting of KMUPs derivative compounds and Sildenafil Analogs compounds.
33. A combination therapy method of Embodiments 31-32, wherein Sildenafil Analogs compounds include Sildenafil, Hydroxyhomosildenafil, Desmethylsildenafil, Acetidenafil, Udenafil, Vardenafil and Homosildenafil.
34. A method of any one of Embodiments 31-32, wherein the statins analogues is one selected from a group consisting of Atorvastatin, Cerivastatin, Fluvastatin, Lovastatin, Mevastatin, Pravastatin, Rosuvastatin, Simvastatin, Pramastatinic acid and its pharmaceutical acceptable salts.
35. A method of any one of Embodiments 31-32, wherein the pharmaceutical composition of a KMUPs derivative and a KMUPs-statins analogues are formulated independently.
36. A method of any one of Embodiments 31-32, wherein the pharmaceutical composition of a KMUPs derivative and a Sildenafil Analogs statins analogues are formulated independently.
37. A method for inhibiting a physiological activity of a lung cell, comprising a step of combination administration of a pharmaceutically effective amount of a compound of a KMUPs analogues and a KMUPs statins analogues to a mammal in need, wherein the compound has a medical effect of preventing of allergic pulmonary vascular remodeling and via NO, suppressed MMP-9 And ICAM-1/VCAM-1 and a combination thereof.
38. A method of Embodiment 37, wherein the pharmaceutical composition comprises:

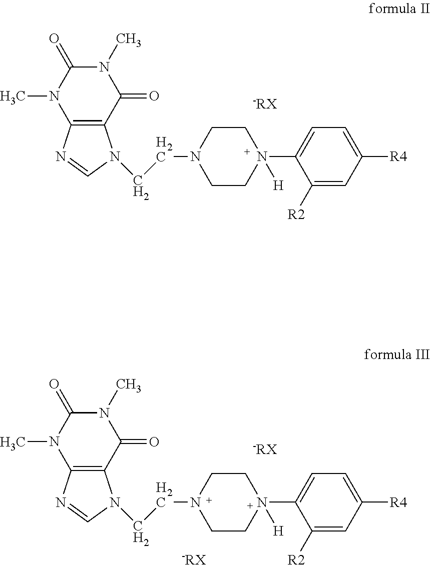

an effective amount of KMUPs complex compound represented by one of formula II and formula III, wherein R₂ and R₄ are each selected independently from the group consisting of a C1˜C5 alkoxy group, a hydrogen; a nitro group, and a halogen atom; and a pharmaceutically acceptable carrier.
39. The method of any one of Embodiments 37-38, wherein the halogen atom is one selected from a group consisting of a fluorine, a chlorine, a bromine and an iodine.
40. A method of any one of Embodiments 37-39, wherein the pharmaceutical composition of the KMUPs derivative compounds is one selected from a group consisting of KMUP-1, KMUP-2, KMUP-3, KMUP-4 and its pharmaceutical acceptable salts.
41. A method of any one of Embodiments 37-40, wherein the pharmaceutical composition of statins analogues is one selected from a group consisting of Atorvastatin, Cerivastatin, Fluvastatin, Lovastatin, Mevastatin, Pravastatin, Rosuvastatin, Simvastatin, Pramastatinic acid and its pharmaceutical acceptable salts.
42. A method of any one of Embodiments 37-41, wherein the pharmaceutical composition of a KMUPs derivative and a statins analogues are formulated independently.
43. A method, comprising steps of providing a subject in need thereof; and administering one selected from a group consisting of Sildenafil Analogs derivative compound and Sildenafil Analogs-RX complex compound, a pharmaceutically acceptable salts thereof; and a pharmaceutical composition thereof to the subject in a dosage from oral 1 to 5.0 milligram per kilogram of body weight of animals.

### REFERENCES

1. C-P Liu, M-S Kuo, B-N Wu, C-Y Chai, Huang H-T, P-W Chung, 1-J Chen. “NO-releasing xanthine KMUP-1 bonded by simvastatin attenuates bleomycin-induced lung inflammation and delayed fibrosis”. Pulm Pharmacol Ther. 2014 February; 27(1):17-28
2. Z-K Dai, T-C Lin, J-C Liou, K-I Cheng, J-Y Chen, L-W Chu, I-J Chen, and B-N Wu et al., “Xanthine-derivative KMUP-1 reduces inflammation and hyperalgesia in a bilateral chronic constriction injury model by suppressing MAPK and NF#B activation”, Mol Pharm. 2014 May 5; 11(5):1621-31.
3. J-L Yeh, C-P Liu, J-H Hsu, C-J Tseng, P-J Wu, Y-Y Wang, J-R Wu, I-J Chen, “KMUP-1 inhibits hypertension-induced left ventricular hypertrophy through regulation of nitric oxide synthases, ERK1/2, and calcineurin”, Kaohsiung Journal of Medical Sciences (2012) 28, 567-576

1. A method for inhibiting a pulmonary inflammation, comprising steps of:
providing a subject in need thereof; and
administering a pharmaceutical composition of a KMUPs complex compound selected from a group consisting of KMUPs-RX complex compound, KMUPs-RX-RX complex compound and administer to the subject a dosage between 1 and 5.0 milligrams per kilogram of body weight,
wherein KMUPs include KMUP-1, KMUP-2, KMUP-3 and KMUP-4:
RX includes a carboxylic group selected from a group consisting of a Statin analogue, a Co-polymer, a poly-γ-polyglutamic acid derivative, ascorbic acid, an oleic acid, a phosphoric acid, a citric acid, a nicotinic acid and a sodium CMC; and
the pulmonary inflammation comprises one selected from a group consisting of a pulmonary artery hypertension, a pulmonary artery proliferation, an allergic pulmonary vascular inflammation, a physiological activity of a lung cell, a vascular remodeling inhibiting disease and a combination thereof.

2. The method as claimed in claim 1, wherein the pulmonary inflammation comprises one selected from a group consisting of an obstructive pulmonary disease, a pulmonary artery hypertension, pulmonary artery proliferation, an allergic pulmonary vascular inflammation, a physiological activity of a lung cell, a vascular remodeling inhibiting disease and a combination thereof.

3. The method as claimed in claim 1, wherein KMUP-1 is 7-[2-[4-(2-chlorobenzene)piperazinyl]ethyl]-1,3-dimethylxanthine, KMUP-2 is 7-[2-[4-(2-methoxybenzene)piperazinyl]ethyl]-1,3-dimethylxanthine, KMUP-3 is 7-[2-[4-(4-nitrobenzene)piperazinyl]ethyl]-1,3-dimethylxanthine and KMUP-4 is 7-[2-[4-(2-nitrobenzene)piperazinyl]ethyl]-1,3-dimethylxanthine.

4. The method as claimed in claim 1, wherein the KMUPs-RX complex compound is one selected from a group consisting of KMUP-1-Atorvastatin complex compound, KMUP-2-Atorvastatin complex compound, KMUP-3-Atorvastatin complex compound, KMUP-4-Atorvastatin complex compound, KMUP-1-Cerivastatin complex compound, KMUP-2-Cerivastatin complex compound, KMUP-3-Cerivastatin complex compound, KMUP-4-Cerivastatin complex; KMUP-1-Fluvastatin complex compound, KMUP-2-Fluvastatin complex compound, KMUP-3-Fluvastatin complex compound, KMUP-4-Fluvastatin complex compound, KMUP-1-Lovastatin complex compound, KMUP-2-Lovastatin complex compound, KMUP-3-Lovastatin complex compound, KMUP-4-Lovastatin complex; KMUP-1-Mevastatin complex compound, KMUP-2-Mevastatin complex compound, KMUP-3-Mevastatin complex compound, KMUP-4-Mevastatin complex compound, KMUP-1-Pravastatin complex compound, KMUP-2-Pravastatin complex compound, KMUP-3-Pravastatin complex compound, KMUP-4-Pravastatin complex compound, KMUP-1-Rosuvastatin complex compound, KMUP-2-Rosuvastatin complex compound, KMUP-3-Rosuvastatin complex compound, KMUP-4-Rosuvastatin complex compound, KMUP-1-Simvastatin complex compound, KMUP-2-Simvastatin complex compound, KMUP-3-Simvastatin complex compound, KMUP-4-Simvastatin complex; KMUP-1-ascorbic acid complex compound, KMUP-2L-ascorbic acid complex compound, KMUP-3L-ascorbic acid complex compound, KMUP-4L-ascorbic acid complex; KMUP-1-phosphoric acid complex compound, KMUP-2-phosphoric acid complex compound, KMUP-3-phosphoric acid complex compound, KMUP-4-phosphoric acid complex; KMUP-1-CMC complex compound, KMUP-2-CMC complex compound, KMUP-3-CMC complex compound, KMUP-4-CMC complex compound, KMUP-1-hyaluronic complex compound, KMUP-2-hyaluronic complex compound, KMUP-3-hyaluronic complex compound, KMUP-4-hyaluronic complex compound, KMUP-1-polyacrylic complex compound, KMUP-2-polyacrylic complex compound, KMUP-3-polyacrylic complex compound, KMUP-4-polyacrylic complex compound, KMUP-1-Eudragit complex compound, KMUP-2-Eudragit complex compound, KMUP-3-Eudragit complex compound, KMUP-4-Eudragit complex; KMUP-1-polylactide complex compound, KMUP-2-polylactide complex compound, KMUP-3-polylactide complex compound, KMUP-4-polylactide complex; KMUP-1-polyglycolic complex compound, KMUP-2-polyglycolic complex compound, KMUP-3-polyglycolic complex compound, KMUP-4-polyglycolic complex compound, KMUP-1-dextran sulfate complex compound, KMUP-2-dextran sulfate complex compound, KMUP-3-dextran sulfate complex compound, KMUP-4-dextran sulfate complex compound, KMUP-1-heparan sulfate complex compound, KMUP-2-heparan sulfate complex compound, KMUP-3-heparan sulfate complex compound, KMUP-4-heparan sulfate complex compound, KMUP-1-alginate complex compound, KMUP-2-alginate complex compound, KMUP-3-alginate complex compound, KMUP-4-alginate complex compound, KMUP-1-γ-PGA complex compound, KMUP-2-γ-PGA complex compound, KMUP-3-γ-PGA complex compound, KMUP-4-γ-PGA complex compound, KMUP-1-APA complex compound, KMUP-2-APA complex compound, KMUP-3-APA complex compound and KMUP-4-APA complex compound.

5. The method as claimed in claim 1, wherein the administration is performed by one selected from an oral, an injection, an inhalation and a topical administration.

6. The method as claimed in claim 5, administering by inhalation or nasal route to a patient in need thereof a therapeutically effective amount of one of a KMUPs complex compound and a pharmaceutical composition in propellant gases.

7. The method as claimed in claim 5, administering by inhalation or nasal route to a patient in need thereof a therapeutically effective amount of one of a KMUPs complex compound and a pharmaceutical composition in dry powder.

8. The method as claimed in claim 5, administering by inhalation or nasal route to a patient in need thereof a therapeutically effective amount of one of a KMUPs complex compound and a pharmaceutical composition in liquid mist inhaler.

9. A combination method for inhibiting a pulmonary inflammation, comprising steps of:
providing a subject suffering from the pulmonary inflammation; and
administering a combination of one selected from a group consisting of Piperazinyl Analogs compounds, and administrating a pharmaceutically effective amount of a compound selected from a group consisting of Statin analogue, a Co-polymer, a poly-γ-polyglutamic acid derivative, ascorbic acid, an oleic acid, a phosphoric acid, a citric acid, a nicotinic acid and a sodium CMC, thereof to the subject in need thereof in a combination dosage between 1 and 5.0 milligrams per kilogram of body weight.

10. The combination method as claimed in claim 9, wherein the pulmonary inflammation comprises one selected from a group consisting of an obstructive pulmonary disease, a pulmonary artery hypertension, pulmonary artery proliferation, an allergic pulmonary vascular inflammation, a physiological activity of a lung cell, a vascular remodeling inhibiting disease and a combination thereof.

11. The combination method as claimed in claim 9, wherein the statins analogues is one selected from a group consisting of Atorvastatin, Cerivastatin, Fluvastatin, Lovastatin, Mevastatin, Pravastatin, Rosuvastatin, Simvastatin, Pramastatinic acid and its pharmaceutical acceptable salts.

12. The combination method as claimed in claim 9, wherein the Co-polymers is one selected from a group consisting of a hyaluronic acid, a polyacrylic acid, a polymethacrylates (PMMA), an Eudragit, a dextran sulfate, a heparan sulfate, a polylactic acid or polylactide (PLA), a polylactic acid sodium (PLA sodium) and a polyglycolic acid sodium (PGA sodium).

13. The combination method as claimed in claim 9, wherein the Poly-γ-polyglutamic acid (γ-PGA) derivative is one selected from a group consisting of an alginate sodium, a poly-γ-polyglutamic acid sodium (γ-PGA sodium), a poly-γ-polyglutamic acid (γ-PGA) and an alginate-poly-lysine-alginate (APA).

14. The combination method as claimed in claim 9, wherein the administration is performed by one selected from an oral, an injection, an inhalation and a topical administration.

15. The combination method as claimed in claim 9, administering by inhalation or nasal route to a patient in need thereof a therapeutically effective amount of one of a Piperazinyl Analogs compound and a pharmaceutical composition in propellant gases.

16. The combination method as claimed in claim 9, administering by inhalation or nasal route to a patient in need thereof a therapeutically effective amount of one of a Piperazinyl Analogs compound and a pharmaceutical composition in dry powder.

17. The combination method as claimed in claim 9, administering by inhalation or nasal route to a patient in need thereof a therapeutically effective amount of one of a Piperazinyl Analogs compound and a pharmaceutical composition in a liquid mist inhaler.

18. The combination method as claimed in claim 9, wherein Piperazinyl Analogs is one selected from a group consisting of KMUPs derivative compounds and Sildenafil Analogs compounds.

19. The combination method as claimed in claim 18 wherein the KMUPs derivative compounds include KMUP-1, KMUP-2, KMUP-3, KMUP-4 and a pharmaceutical acceptable salts and a combination thereof.

20. The combination method as claimed in claim 18, wherein Sildenafil Analogs compounds include Sildenafil, Hydroxyhomosildenafil, Desmethylsildenafil, Acetidenafil, Udenafil, Vardenafil and Homosildenafil.
